(19) United States
(12) Patent Application Publication
(10) Pub. No.: US 2026/0264078 A1
(43) Pub. Date: Sep. 10, 2026

(54) SYSTEMS AND METHODS FOR PROCESSING ANALYTES

(71) Applicant: ULTIMA GENOMICS, INC., Fremont, CA (US)

(72) Inventors: Nathan BECKETT, Oakland, CA (US); Nathan CASWELL, Sunnyvale, CA (US); Stephanie KUBECKA, San Jose, CA (US)

(21) Appl. No.: 19/569,858

(22) Filed: Mar. 17, 2026

Related U.S. Application Data
(63) Continuation of application No. PCT/US2024/048160, filed on Sep. 24, 2024.
(60) Provisional application No. 63/585,060, filed on Sep. 25, 2023.

(51) Int. Cl. B01L 3/00

(52) U.S. Cl. CPC B01L 3/50273; B01L 2200/0647; B01L 2200/141; B01L 2200/16; B01L 2300/041; B01L 2400/049

(57) ABSTRACT

Provided herein are systems and methods for processing analytes. Such systems and methods for processing analytes include, for example, systems and methods for sequencing nucleic acid samples. Analytes may be processed on an open substrate. Substrates may be supported from a bottom surface or a top surface, such as in a cantilever configuration.

### CROSS-REFERENCE

This application is a continuation of International Application No. PCT/US2024/048160, filed Sep. 24, 2024, which claims the benefit of U.S. Provisional Patent Application No. 63/585,060, filed Sep. 25, 2023, which is entirely incorporated by reference herein.

### BACKGROUND

Biological sample processing has various applications in the fields of molecular biology and medicine (e.g., diagnosis). For example, nucleic acid sequencing may provide information that may be used to diagnose a certain condition in a subject and in some cases tailor a treatment plan. Sequencing is widely used for molecular biology applications, including vector designs, gene therapy, vaccine design, industrial strain design and verification. Biological sample processing may involve a fluidics system and/or a detection system.

### SUMMARY

Provided herein are systems and methods for processing analytes, such as sequencing nucleic acid samples. Analytes may be processed on an open substrate. Substrates may be supported from a bottom surface or a top surface, such as in a cantilever configuration. The present disclosure may be advantageous to improve sequencing results.

In an aspect, provided is a system for processing analytes, comprising: a chamber; a lid plate, wherein the lid plate is configured to close and open with respect to the chamber, wherein the chamber and lid plate enclose a sample environment configured to receive a substrate therein; a detector that penetrates the lid plate into the sample environment; a fluid dispenser that penetrates the lid plate into the sample environment; and a chuck that penetrates the lid plate into the sample environment, wherein the chuck is configured to affix to a top surface of the substrate to support the substrate in the sample environment.

In some embodiments, the chuck is configured to rotate the substrate within the sample environment.

In some embodiments, the chuck is a vacuum chuck.

In some embodiments, an inner surface of the chamber comprises one or more nozzles fluidically coupled to a hot fluid supply and a cold fluid supply, wherein hot fluid in the hot fluid supply is maintained at temperatures or temperature ranges that are higher than those of cold fluid in the cold fluid supply, wherein the one or more nozzles are configured to direct cold fluid, hot fluid, or both onto a bottom surface of the substrate.

In some embodiments, an inner surface of the chamber comprises one or more additional nozzles fluidically coupled to a cleaning solution, wherein the cleaning solution is configured to direct the cleaning solution to a bottom surface of the lid plate.

In some embodiments, the chamber comprises a drainage outlet.

In some embodiments, the system further comprises a mechanical arm coupled to the chamber, wherein the mechanical arm is configured to move the chamber relative to the lid plate to adjust the chamber between an open configuration and closed configuration.

In some embodiments, the system further comprises a substrate handler, wherein the substrate handler is configured to load or unload the substrate onto the chuck in the sample environment in the open configuration.

In some embodiments, the system further comprises a container comprising exterior walls enclosing the chamber, the lid plate, the detector, the fluid dispenser, and the chuck.

In some embodiments, one or more walls of the container comprises a user interface. In some embodiments, the user interface comprises one or more of a display and a speaker.

In some embodiments, one or more walls of the container comprises a substrate slot configured to permit the substrate to enter or exit the container.

In some embodiments, one or more walls of the container comprises a reagent slot configured to permit a reagent cartridge to enter or exit the container.

In some embodiments, a maximum height of the container is at most 1.5 meters (m).

In some embodiments, a maximum depth of the container is at most 1.0 meters (m).

In some embodiments, the fluid dispenser is configured to dispense fluid to the top surface of the substrate.

In some embodiments, the detector is configured to collect one or more signals from the top surface of the substrate.

In some embodiments, the system further comprises the substrate, wherein the substrate comprises on the top surface a plurality of analytes immobilized thereon.

In some embodiments, the plurality of analytes comprises a plurality of nucleic acid molecules.

In some embodiments, the plurality of nucleic acid molecules is directly immobilized to the top surface.

In some embodiments, the plurality of nucleic acid molecules is coupled to a plurality of beads, wherein the plurality of beads are immobilized to the top surface.

In some embodiments, the substrate is substantially planar.

In some embodiments, the substrate is unpatterned.

In some embodiments, the substrate is patterned.

In some embodiments, the system further comprises one or more processors operably connected to the fluid dispenser, the detector, and the chuck, and individually or collectively programmed to rotate the substrate via the chuck, dispense fluid via the fluid dispenser, or collect signals from the substrate via the detector.

In some embodiments, the one or more processors are operably connected to one or more mechanical arms and configured to move at least one mechanical arm of the one or more mechanical arms along at least one axis, wherein the one or more mechanical arms are connected to the lid plate or chamber or both.

In another aspect, provided is a method for processing analytes, comprising: loading a substrate onto a chuck in a sample environment through an opening between a chamber and a lid plate, such that the chuck is affixed to a top surface of the substrate and supports the substrate, wherein the substrate comprises a plurality of analytes immobilized to the top surface; closing the opening between the chamber and the lid plate; and processing the plurality of analytes on the top surface of the substrate by operating one or more of a fluid dispenser and a detector, wherein the fluid dispenser and the detector penetrate the lid plate into the sample environment.

In some embodiments, the plurality of analytes comprises a plurality of nucleic acid molecules.

In some embodiments, the plurality of nucleic acid molecules is directly immobilized to the top surface.

In some embodiments, the plurality of nucleic acid molecules is coupled to a plurality of beads, wherein the plurality of beads is immobilized to the top surface.

In some embodiments, the processing in (c) comprises sequencing the plurality of nucleic acid molecules, or derivatives thereof.

In some embodiments, the derivatives comprise amplification products of the plurality of nucleic acid molecules.

In some embodiments, the processing in (c) comprises amplifying the plurality of nucleic acid molecules on the top surface to generate amplification products immobilized to the top surface. In some embodiments, the method further comprises sequencing the amplification products.

In some embodiments, the amplifying comprises polymerase chain reaction (PCR), recombinase polymerase amplification (RPA), rolling circle amplification (RCA), or multiple displacement amplification (MDA).

In some embodiments, the method further comprises during the processing of the plurality of analytes in (c), thermocycling the temperature of the substrate between at least two different temperature or temperature ranges.

In some embodiments, the thermocycling comprises directing cold fluid, hot fluid, or both from one or more nozzles disposed on an inner surface of the chamber to a bottom surface of the substrate, wherein the one or more nozzles are fluidically coupled to a hot fluid supply and a cold fluid supply, wherein the hot fluid in the hot fluid supply is maintained at temperatures or temperature ranges that are higher than those of the cold fluid in the cold fluid supply.

Another aspect of the present disclosure provides a system comprising one or more computer processors and computer memory coupled thereto. The computer memory comprises machine executable code that, upon execution by the one or more computer processors, implements any of the methods above or elsewhere herein. Another aspect of the present disclosure provides a non-transitory computer readable medium comprising machine executable code that, upon execution by one or more computer processors, implements any of the methods above or elsewhere herein.

Additional aspects and advantages of the present disclosure will become readily apparent to those skilled in this art from the following detailed description, wherein only illustrative instances of the present disclosure are shown and described. As will be realized, the present disclosure is capable of other and different instances, and its several details are capable of modifications in various obvious respects, all without departing from the disclosure.

Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings (also “Figure” and “FIG.” herein) of which:

FIG. 1 illustrates an example workflow for processing a sample for sequencing.

FIG. 2 illustrates examples of individually addressable locations distributed on substrates, as described herein.

FIG. 3 shows an example image of a substrate with a hexagonal lattice of beads, as described herein.

FIG. 4 illustrates example systems and methods for loading a sample or a reagent onto a substrate, as described herein.

FIGS. 5A-5B illustrate multiplexed stations in a sequencing system.

FIG. 6 illustrates a computer system that is programmed or otherwise configured to implement methods provided herein.

FIG. 7A illustrates example cantilever configurations of an open substrate system.

FIGS. 7B and 7C illustrate the sample environment system in the cantilever configuration in the closed and open position, respectively.

FIG. 7D illustrates a schematic top view of lid plate.

FIG. 7E and FIG. 7F illustrate a schematic front view and cross-sectional side view of a packaged system, e.g., of a sequencing system.

FIG. 8A shows different active surface areas on an open substrate.

FIG. 8B illustrates different active areas that have distinct surface chemistry.

FIGS. 9A-9D show different configurations of chemistry and detection stations of the open substrate systems in the cantilever configuration.

FIGS. 10A-10C illustrate different schematics of using a physical scraper, such as a doctor blade tool, a mop tool, or a rigid object tool, to disperse reagents across different regions of a substrate.

FIG. 10D illustrate different schematics of using inkjet heads, multi-needle dispensers, single-needle dispensers, and actuated dispensers.

FIG. 10E illustrate different schematics of using slot die tools to dispense fluids to a substrate.

### DETAILED DESCRIPTION

While various embodiments of the invention have been shown and described herein, it will be obvious to those skilled in the art that such embodiments are provided by way of example only. Numerous variations, changes, and substitutions may occur to those skilled in the art without departing from the invention. It should be understood that various alternatives to the embodiments of the invention described herein may be employed.

As used herein, the singular forms “a,” “an,” and “the” include the plural reference unless the context clearly dictates otherwise.

When a range of values is provided, it is to be understood that each intervening value between the upper and lower limit of that range, and any other stated or intervening value in that stated range is encompassed within the scope of the present disclosure. Where the stated range includes upper or lower limits, ranges excluding either of those included limits are also included in the present disclosure.

The term “analyte,” as used herein, generally refers to an object that is the subject of analysis, or an object, regardless of being the subject of analysis, that is directly or indirectly analyzed during a process. An analyte may be synthetic. An analyte may be, originate from, and/or be derived from, a sample, such as a biological sample. In some examples, an analyte is or includes a molecule, macromolecule (e.g., nucleic acid, carbohydrate, protein, lipid, etc.), nucleic acid, carbohydrate, lipid, antibody, antibody fragment, antigen, peptide, polypeptide, protein, macromolecular group (e.g., glycoproteins, proteoglycans, ribozymes, liposomes, etc.), cell, tissue, biological particle, or an organism, or any engineered copy or variant thereof, or any combination thereof. The term “processing an analyte,” as used herein, generally refers to one or more stages of interaction with one more samples. Processing an analyte may comprise conducting a chemical reaction, biochemical reaction, enzymatic reaction, hybridization reaction, polymerization reaction, physical reaction, any other reaction, or a combination thereof with, in the presence of, or on, the analyte. Processing an analyte may comprise physical and/or chemical manipulation of the analyte. For example, processing an analyte may comprise detection of a chemical change or physical change, addition of or subtraction of material, atoms, or molecules, molecular confirmation, detection of the presence of a fluorescent label, detection of a Forster resonance energy transfer (FRET) interaction, or inference of absence of fluorescence.

The term “biological sample,” as used herein, generally refers to any sample derived from a subject or specimen. The biological sample can be a fluid, tissue, collection of cells (e.g., cheek swab), hair sample, or feces sample. The fluid can be blood (e.g., whole blood), saliva, urine, or sweat. The tissue can be from an organ (e.g., liver, lung, or thyroid), or a mass of cellular material, such as, for example, a tumor. The biological sample can be a cellular sample or cell-free sample. Examples of biological samples include nucleic acid molecules, amino acids, polypeptides, proteins, carbohydrates, fats, or viruses. In an example, a biological sample is a nucleic acid sample including one or more nucleic acid molecules, such as deoxyribonucleic acid (DNA) and/or ribonucleic acid (RNA). The nucleic acid sample may comprise cell-free nucleic acid molecules, such as cell-free DNA or cell-free RNA. Non-limiting examples of nucleic acids include DNA, RNA, genomic DNA or synthetic DNA/RNA or coding or non-coding regions of a gene or gene fragment, loci (locus) defined from linkage analysis, exons, introns, messenger RNA (mRNA), transfer RNA, ribosomal RNA (rRNA), short interfering RNA (siRNA), short-hairpin RNA (shRNA), micro-RNA (miRNA), ribozymes, cDNA, recombinant nucleic acids, branched nucleic acids, plasmids, vectors, isolated DNA of any sequence, and isolated RNA of any sequence. Further, samples may be extracted from variety of animal fluids containing cell free sequences, including but not limited to blood, serum, plasma, vitreous, sputum, urine, tears, perspiration, saliva, semen, mucosal excretions, mucus, spinal fluid, amniotic fluid, lymph fluid and the like. Cell free polynucleotides may be fetal in origin (via fluid taken from a pregnant subject) or may be derived from tissue of the subject itself. A biological sample may also refer to a sample engineered to mimic one or more properties (e.g., nucleic acid sequence properties, e.g., sequence identity, length, GC content, etc.) of a sample derived from a subject or specimen.

As used herein, the term “template nucleic acid” generally refers to the nucleic acid to be sequenced. The template nucleic acid may be an analyte or be associated with an analyte. For example, the analyte can be a mRNA, and the template nucleic acid is the mRNA or a cDNA derived from the mRNA, or other derivative thereof. In another example, the analyte can be a protein, and the template nucleic acid is an oligonucleotide that is conjugated to an antibody that binds to the protein, or derivative thereof. Examples of sequencing include single molecule sequencing or sequencing by synthesis, for example. Sequencing may comprise generating sequencing signals and/or sequencing reads. Sequencing may be performed on template nucleic acids immobilized on a support, such as a flow cell, substrate, and/or one or more beads. In some cases, a template nucleic acid may be amplified to produce a colony of nucleic acid molecules attached to the support to produce amplified sequencing signals. In one example, (i) a template nucleic acid is subjected to a nucleic acid reaction, e.g., amplification, to produce a clonal population of the nucleic acid attached to a bead, the bead immobilized to a substrate, (ii) amplified sequencing signals from the immobilized bead are detected from the substrate surface during or following one or more nucleotide flows, and (iii) the sequencing signals are processed to generate sequencing reads. The substrate surface may immobilize multiple beads at distinct locations, each bead containing distinct colonies of nucleic acids, and upon detecting the substrate surface, multiple sequencing signals may be simultaneously or substantially simultaneously processed from the different immobilized beads at the distinct locations to generate multiple sequencing reads. In some sequencing methods, the nucleotide flows comprise non-terminated nucleotides. In some sequencing methods, the nucleotide flows comprise terminated nucleotides.

The term “nucleotide,” as used herein, generally refers to any nucleotide or nucleotide analog. The nucleotide may be naturally occurring or non-naturally occurring. The nucleotide may be a non-standard, modified, synthesized, or engineered nucleotide. The nucleotide may include a canonical base or a non-canonical base. The nucleotide may comprise an alternative base. The nucleotide may include a modified polyphosphate chain (e.g., triphosphate coupled to a fluorophore). The nucleotide may comprise a label. The nucleotide may be terminated (e.g., reversibly terminated). The nucleotide may be non-terminated (e.g., natural or modified). In some cases, nucleotides may include modifications in their phosphate moieties, including modifications to a triphosphate moiety. Nucleic acids may also be modified at the base moiety (e.g., at one or more atoms that typically are available to form a hydrogen bond with a complementary nucleotide and/or at one or more atoms that are not typically capable of forming a hydrogen bond with a complementary nucleotide), sugar moiety or phosphate backbone. Nucleic acids may also contain amine-modified groups, such as aminoallyl-dUTP (aa-dUTP) and aminohexhylacrylamide-dCTP (aha-dCTP) to allow covalent attachment of amine reactive moieties, such as N-hydroxysuccinimide esters (NHS). Alternatives to standard DNA base pairs or RNA base pairs in the oligonucleotides of the present disclosure can provide higher density in bits per cubic mm, higher safety (resistant to accidental or purposeful synthesis of natural toxins), easier discrimination in photo-programmed polymerases, or lower secondary structure. Nucleotides may be capable of reacting or bonding with detectable moieties for detection.

The term “sequencing,” as used herein, generally refers to a process for generating or identifying a sequence of a biological molecule, such as a nucleic acid. The sequence may be a nucleic acid sequence which comprises a sequence of nucleic acid bases. Examples of sequencing include single molecule sequencing or sequencing by synthesis, for example. Sequencing may comprise generating sequencing signals and/or sequencing reads.

### Sample Processing Methods

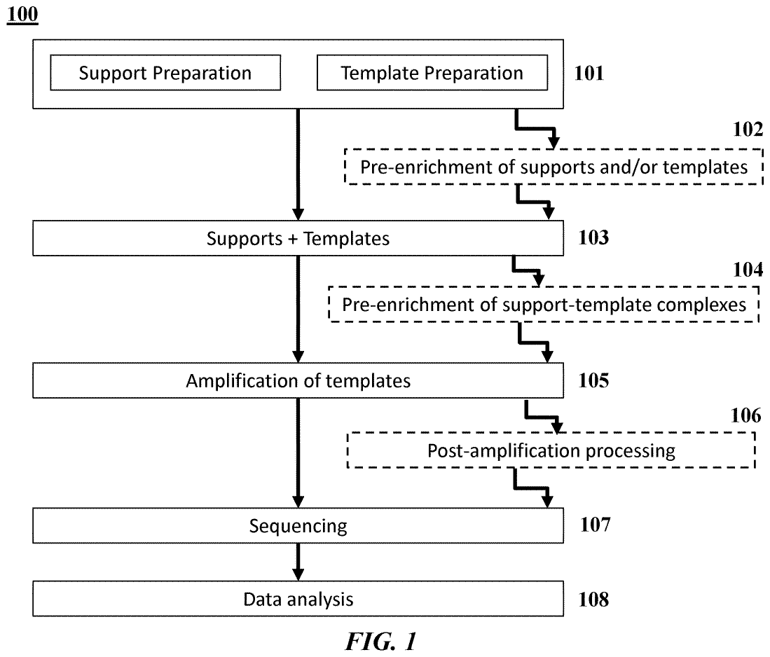

Described herein are devices, systems, methods, compositions, and kits for processing samples, such as to prepare a sample for sequencing, to sequence a sample, and/or to analyze sequencing data. FIG. 1 illustrates an example sequencing workflow 100, according to the devices, systems, methods, compositions, and kits of the present disclosure.

Supports and/or template nucleic acids may be provided and/or prepared (101) to be compatible with downstream sequencing operations (e.g., 107). A support (e.g., bead) may help immobilize a template nucleic acid to a substrate, such as when the template nucleic acid is coupled to the support, and the support is in turn immobilized to the substrate. The support may further function as a binding entity to retain derivatives molecules (e.g., amplification products) from a same template nucleic acid together for downstream processing, such as for sequencing operations. This may be useful in distinguishing a colony from other colonies (e.g., on other supports) and generating amplified sequencing signals corresponding to a template nucleic acid. A support may comprise an oligonucleotide comprising one or more functional nucleic acid sequences. The oligonucleotide may be single-stranded, double-stranded, or partially double-stranded. For example, the oligonucleotide may comprise a capture sequence, a primer sequence, a sequencing primer sequence, a barcode sequence, a sample index sequence, a unique molecular identifier (UMI), a flow cell adapter sequence, an adapter sequence, a target sequence, a random sequence, a binding sequence (e.g., for a splint, primer, template nucleic acid, capture sequence, etc.), or any other functional sequence useful for a downstream operation, a complement thereof, or any combination thereof. The capture sequence may be configured to hybridize to a sequence of a template nucleic acid or derivative thereof. The support may comprise a plurality of oligonucleotides, for example on the order of 10, 10², 10³, 10⁴, 10⁵, 10⁶, 10⁷, or more molecules. The support may comprise a single species of oligonucleotide which comprise identical sequences. The support may comprise multiple species of oligonucleotides which have varying sequences. In some cases, the support comprises a single species of a primer (e.g., forward primer) for amplification. In some cases, the support comprises two species of primer (e.g., forward primer, reverse primer) for amplification. Devices, systems, methods, compositions, and kits for preparing and using support species are described in further detail in U.S. Patent Pub. No. 20220042072A1 and International Patent Pub. No. WO2022040557A2, each of which is entirely incorporated herein by reference for all purposes.

A support may comprise one or more capture entities, where a capture entity is configured for capture by a capturing entity. A capture entity may be coupled to or be part of an oligonucleotide coupled to the support. A capture entity may be coupled to or be part of the support. Examples of capture entity-capturing entity pairs and capturing entity-capture entity pairs include: streptavidin (SA)-biotin; complementary sequences; magnetic particle-magnetic field system; charged particle-electric field system; azide-cyclooctyne; thiol-maleimide; click chemistry pairs; cross-linking pairs; etc. The capture entity-capturing entity pair may comprise one or more chemically modified bases. A capture entity and capturing entity may bind, couple, hybridize, or otherwise associate with each other. The association may comprise formation of a covalent bond, non-covalent bond, releasable bond (e.g., cleavable bond that is cleavable upon application of a stimulus), and/or no bond. The capture entity may be capable of linking to a nucleotide. In some instances, the capturing entity may comprise a secondary capture entity, for example, for subsequent capture by a secondary capturing entity. The secondary capture entity and secondary capturing entity may comprise any one or more of the capturing mechanisms described elsewhere herein.

A support may comprise one or more cleavable moieties, also referred to herein as excisable moieties. The cleavable moiety may be coupled to or be part of an oligonucleotide coupled to the support. The cleavable moiety may be coupled to the support. A cleavable moiety may comprise any useful moiety that can be used to cleave an oligonucleotide (or portion thereof) from the support, or otherwise release a nucleic acid strand from the support and/or the oligonucleotide. A cleavable moiety may comprise a uracil, a ribonucleotide, methylated nucleotide, or other modified nucleotide that is excisable or cleavable using an enzyme (e.g., UDG, RNAse, APE1, MspJI, endonuclease, exonuclease, etc.). The cleavable moiety may comprise an abasic site or an analog of an abasic site (e.g., dSpacer), a dideoxyribose, a spacer, e.g., C3 spacer, hexanediol, triethylene glycol spacer (e.g., Spacer 9), hexa-ethyleneglycol spacer (e.g., Spacer 18), a photocleavable moiety, or combinations or analogs thereof. Alternatively, or in addition, the cleavable moiety may be cleavable using one or more stimuli, e.g., photo-stimulus, chemical stimulus, thermal stimulus, etc.

The sequencing workflow 100 may not involve supports, for example when a template nucleic acid and/or its derivatives are directly attached to a substrate and amplified and/or sequenced from the substrate.

A template nucleic acid may include an insert sequence sourced from a biological sample. The template nucleic acid may be derived from any nucleic acid of the biological sample (e.g., endogenous nucleic acid) and result from any number of processing operations, such as but not limited to fragmentation, degradation or digestion, transposition, ligation, reverse transcription, extension, replication, etc. The template nucleic acid may be single-stranded, double-stranded, or partially double-stranded. A template nucleic acid may comprise one or more functional nucleic acid sequences. For example, the template nucleic acid may comprise a capture sequence, a primer sequence, a sequencing primer sequence, a barcode sequence, a sample index sequence, a unique molecular identifier (UMI), a flow cell adapter sequence, an adapter sequence, a target sequence, a random sequence, a binding sequence (e.g., for a splint, primer, template nucleic acid, capture sequence, etc.), or any other functional sequence useful for a downstream operation, a complement thereof, or any combination thereof. The template nucleic acid may comprise an adapter sequence configured to be captured by a capture sequence of an oligonucleotide coupled to a support. The one or more functional nucleic acid sequences may be disposed at one end or both ends of the insert sequence. A nucleic acid molecule comprising the insert sequence, or complement thereof, may be processed with (e.g., attached to, extend from, etc.) one or more adapter molecules to generate the template nucleic acid comprising the insert sequence and one or more functional nucleic acid sequences. A template nucleic acid may comprise one or more capture entities and/or one or more cleavable moieties that are described elsewhere herein.

Optionally, the supports and/or template nucleic acids may be pre-enriched (102). For example, a support comprising a distinct oligonucleotide sequence is pre-enriched to isolate from a mixture comprising support(s) that do not have the distinct oligonucleotide sequence. For example, a template nucleic acid comprising a distinct configuration (e.g., comprising a particular adapter sequence) is pre-enriched to isolate from a mixture comprising template nucleic acids that do not have the distinct configuration. In some cases, the capture entity on the supports and/or template nucleic acids are used for pre-enrichment.

The supports and template nucleic acids may be attached (103) to generate support-template complexes. A template nucleic acid may be coupled to a support via any method(s) that results in a stable association between the template nucleic acid and the support. For example, the template nucleic acid may hybridize to an oligonucleotide on the support; the template nucleic acid may be ligated to a nucleic acid coupled to the support; the template nucleic acid may hybridize to one or more intermediary molecules, such as a splint, bridge, and/or primer molecule, which hybridizes to an oligonucleotide on the support; and/or the template nucleic acid may be hybridized to an oligonucleotide on a support, which oligonucleotide comprises a primer sequence which is extended. In some cases, the respective concentrations of the supports and template nucleic acids may be adjusted such that a majority of support-template complexes are single template-attached supports (e.g., a support attached to a single template nucleic acid).

Optionally, support-template complexes may be pre-enriched (104), wherein a support-template complex is isolated from a mixture comprising support(s) and/or template nucleic acid(s) that are not attached to each other. In some cases, the capture entity on the supports and/or template nucleic acids are used for pre-enrichment.

The template nucleic acids may be subjected to amplification reactions (105) to generate a plurality of amplification products immobilized to the support. Such amplification reactions may comprise performing polymerase chain reaction (PCR) or any other amplification methods described herein, including but not limited to emulsion PCR (ePCR or emPCR), isothermal amplification, recombinase polymerase amplification (RPA), rolling circle amplification (RCA), multiple displacement amplification (MDA), bridge amplification, template walking, etc. Amplification reactions can occur while the support is immobilized to a substrate. Amplification reactions can occur off the substrate, such as in solution, or on a different surface or platform. Amplification reactions can occur in isolated reaction volumes, such as within multiple droplets in an emulsion during emulsion PCR (ePCR or emPCR), or in wells or tubes.

Optionally, subsequent to amplification, the supports, template nucleic acids, and/or support-template complexes may be subjected to post-amplification processing (106). Often, subsequent to amplification, a resulting mixture may comprise a mix of positive supports (e.g., those comprising a template nucleic acid molecule) and negative supports (e.g., those not attached to template nucleic acid molecules). Enrichment procedure(s) may isolate positive supports from the mixtures. Example methods of enrichment of amplified supports are described in U.S. Pat. No. 10,900,078, U.S. Patent Pub. No. 20210079464A1, and International Patent Pub. No. WO2022040557A2, each of which is entirely incorporated by reference herein.

The template nucleic acids may be subject to sequencing (107). The template nucleic acid(s) may be sequenced while attached to the support. Alternatively, the template nucleic acid molecules may be free of the support when sequenced and/or analyzed. The template nucleic acids may be sequenced while immobilized to a substrate, such as via a support or otherwise. Examples of substrate-based sample processing systems are described elsewhere herein. Any sequencing method may be used, for example pyrosequencing, single molecule sequencing, sequencing by synthesis (SBS), sequencing by ligation, sequencing by binding, etc.

For example, sequencing comprises extending a sequencing primer (or growing strand) hybridized to a template nucleic acid by providing labeled nucleotide reagents, washing away unincorporated nucleotides from the reaction space, and detecting one or more signals from the labeled nucleotide reagents which are indicative of an incorporation event or lack thereof. After detection, the labels may be cleaved and the whole process may be repeated any number of times to determine sequence information of the template nucleic acid. One or more intermediary flows may be provided intra- or inter-repeat, such as washing flows, label cleaving flows, terminator cleaving flows, reaction-completing flows (e.g., double tap flow, triple tap flow, etc.), labeled flows (or bright flows), unlabeled flows (or dark flows), phasing flows, chemical scar capping flows, etc. A nucleotide mixture that is provided during any one flow may comprise only labeled nucleotides, only unlabeled nucleotides, or a mixture of labeled and unlabeled nucleotides. The mixture of labeled and unlabeled nucleotides may be of any fraction of labeled nucleotides, such as at least or at most 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, or 99%. A nucleotide mixture that is provided during any one flow may comprise only non-terminated nucleotides, only terminated nucleotides, or a mixture of terminated and non-terminated nucleotides. When using only non-terminated nucleotides, terminator cleaving flows may be omitted from the sequencing process. When using terminated nucleotides, to proceed with the next step of extension, prior to, during, or subsequent to detection, a terminator cleaving flow may be provided to cleave blocking moieties. A nucleotide mixture that is provided during any one flow may comprise any number of canonical base types (e.g., A, T, G, C, U), such as a single canonical base type, two canonical base types, three canonical base types, four canonical base types or five canonical base types (including T and U). Different types of nucleotide bases may be flowed in any order and/or in any mixture of base types that is useful for sequencing. Various flow-based sequencing systems and methods are described in U.S. Pat. Pub. No. 2022/0170089A1, which is entirely incorporated by reference herein for all purposes. Labeled nucleotides may comprise a dye, fluorophore, or quantum dot, multiples thereof, and/or combination thereof. In some cases, nucleotides of different canonical base types may be labeled and detectable at a single frequency (e.g., using the same or different dyes). In other cases, nucleotides of different canonical base types may be labeled and detectable at different frequencies (e.g., using the same or different dyes).

Subsequent to sequencing, the sequencing signals collected and/or generated may be subjected to data analysis (108). The sequencing signals may be processed to generate base calls and/or sequencing reads. In some cases, the sequencing reads may be processed to generate diagnostics data to the biological sample, or the subject from which the biological sample was derived from. The data analysis may comprise image processing, alignment to a genome or reference genome, training and/or trained algorithms, error correction, and the like.

While the sequencing workflow 100 with respect to FIG. 1 has been described with respect to the use of supports to bind template molecules, it will be appreciated that the different supports may be effectively replaced by using spatially distinct locations on one or more surfaces, which do not necessarily have to be the surfaces of individual supports (e.g., beads). For example, a first spatially distinct location on a surface may be capable of directly immobilizing a first colony of a first template nucleic acid and a second spatially distinct location on the same surface (or a different surface) may be capable of directly immobilizing a second colony of a second template nucleic acid to distinguish from the first colony. In some cases, the surface comprising the spatially distinct locations may be a surface of the substrate on which the sample is sequenced, thus streamlining the amplification-sequencing workflow.

It will be appreciated that in some instances, the different operations described in the sequencing workflow 100 may be performed in a different order. It will be appreciated that in some instances, one or more operations described in the sequencing workflow 100 may be omitted or replaced with other comparable operation(s). It will be appreciated that in some instances, one or more additional operations described in the sequencing workflow 100 may be performed. The different operations described with respect to sequencing workflow 100 may be performed with the help of open substrate systems described herein.

### Open Substrate Systems

Described herein are devices, systems, and methods that use open substrates or open flow cell geometries to process a sample. The term “open substrate,” as used herein, generally refers to a substrate in which any point on an active surface of the substrate is physically accessible from a direction normal to the substrate. The devices, systems and methods may be used to facilitate any application or process involving a reaction or interaction between two objects, such as between an analyte and a reagent or between two reagents. For example, the reaction or interaction may be chemical (e.g., polymerase reaction) or physical (e.g., displacement). The devices, systems, and methods described herein may benefit from higher efficiency, such as from faster reagent delivery and lower volumes of reagents required per surface area. The devices, systems, and methods described herein may avoid contamination problems common to microfluidic channel flow cells that are fed from multiport valves which can be a source of carryover from one reagent to the next. The devices, systems, and methods may benefit from shorter completion time, use of fewer resources (e.g., various reagents), and/or reduced system costs. The open substrates or flow cell geometries may be used to process any analyte from any sample, such as but not limited to, nucleic acid molecules, protein molecules, antibodies, antigens, cells, and/or organisms, as described herein. The open substrates or flow cell geometries may be used for any application or process, such as, but not limited to, sequencing by synthesis, sequencing by ligation, amplification, proteomics, single cell processing, barcoding, and sample preparation, as described herein.

A sample processing system may comprise a substrate, and devices and systems that perform one or more operations with or on the substrate. The sample processing system may permit highly efficient dispensing of analytes and reagents onto the substrate. The sample processing may permit highly efficient imaging of one or more analytes, or signals corresponding thereto, on the substrate. The sample processing system may comprise an imaging system comprising a detector. Substrates, detectors, and sample processing hardware that can be used in the sample processing system are described in further detail in U.S. Patent Pub. No. 20200326327A1, U.S. Patent Pub. No. 20210079464A1, International Patent Pub. No. WO2022072652A1, and U.S. Patent Pub. No. 20210354126A1, each of which is entirely incorporated herein by reference for all purposes.

### Substrates

The substrate may be a solid substrate. The substrate may entirely or partially comprise one or more of rubber, glass, silicon, a metal such as aluminum, copper, titanium, chromium, or steel, a ceramic such as titanium oxide or silicon nitride, a plastic such as polyethylene (PE), low-density polyethylene (LDPE), high-density polyethylene (HDPE), polypropylene (PP), polystyrene (PS), high impact polystyrene (HIPS), polyvinyl chloride (PVC), polyvinylidene chloride (PVDC), acrylonitrile butadiene styrene (ABS), polyacetylene, polyamides, polycarbonates, polyesters, polyurethanes, polyepoxide, polymethyl methacrylate (PMMA), polytetrafluoroethylene (PTFE), phenol formaldehyde (PF), melamine formaldehyde (MF), urea-formaldehyde (UF), polyetheretherketone (PEEK), polyetherimide (PEI), polyimides, polylactic acid (PLA), furans, silicones, polysulfones, any mixture of any of the preceding materials, or any other appropriate material. The substrate may be entirely or partially coated with one or more layers of a metal such as aluminum, copper, silver, or gold, an oxide such as a silicon oxide (SiₓO_{y}, where x, y may take on any possible values), a photoresist such as SU8, a surface coating such as an aminosilane or hydrogel, polyacrylic acid, polyacrylamide dextran, polyethylene glycol (PEG), or any combination of any of the preceding materials, or any other appropriate coating. The substrate may comprise multiple layers of the same or different type of material. The substrate may be fully or partially opaque to visible light. The substrate may be fully or partially transparent to visible light. A surface of the substrate may be modified to comprise active chemical groups, such as amines, esters, hydroxyls, epoxides, and the like, or a combination thereof. A surface of the substrate may be modified to comprise any of the binders or linkers described herein. In some instances, such binders, linkers, active chemical groups, and the like may be added as an additional layer or coating to the substrate.

The substrate may have the general form of a cylinder, a cylindrical shell or disk, a rectangular prism, or any other geometric form. The substrate may have a thickness (e.g., a minimum dimension) of at least and/or at most about 100 micrometers (μm), 200 μm, 500 μm, 1 millimeter (mm), 2 mm, 5 mm, 10 mm, 15 mm, 20 mm, 25 mm, 30 mm, 35 mm, 40 mm, 45 mm, 50 or mm. The substrate may have a first lateral dimension (such as a width for a substrate having the general form of a rectangular prism or a radius or diameter for a substrate having the general form of a cylinder) and/or a second lateral dimension (such as a length for a substrate having the general form of a rectangular prism) of at least and/or at most about 1 mm, 2 mm, 5 mm, 10 mm, 20 mm, 30 mm, 40 mm, 50 mm, 100 mm, 150 mm, 200 mm, 300 mm, 400 mm, 500 mm, 1,000 mm, 1,500 mm, 2,000 mm, 2,500 mm, 3,000 mm, 4,000 mm, 5,000 mm or more.

One or more surfaces of the substrate may be exposed to and accessible from a surrounding open environment. In some cases, the surrounding open environment may be controlled and/or confined in a larger controlled environment.

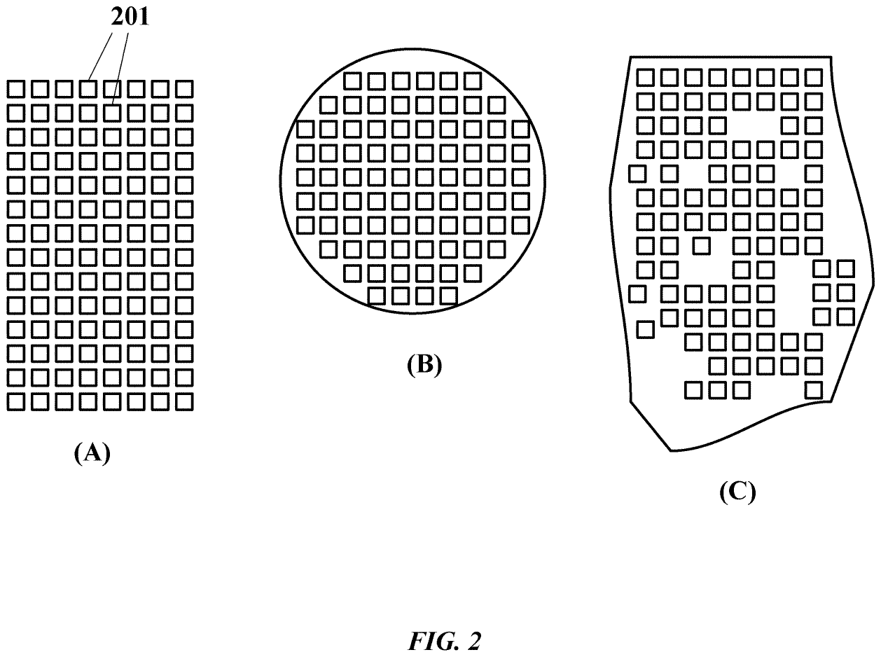

The substrate may comprise a plurality of individually addressable locations. The individually addressable locations may comprise locations that are physically accessible for manipulation. The manipulation may comprise, for example, placement, extraction, reagent dispensing, seeding, heating, cooling, or agitation. The manipulation may be accomplished through, for example, localized microfluidic, pipet, optical, laser, acoustic, magnetic, and/or electromagnetic interactions with the analyte or its surroundings. The individually addressable locations may comprise locations that are digitally accessible. For example, each individually addressable location may be located, identified, and/or accessed electronically or digitally for indexing, mapping, sensing, associating with a device (e.g., detector, processor, dispenser, etc.), or otherwise processing. In some cases, the individually addressable locations may be defined by physical features of the substrate (e.g., on a modified surface) to distinguish them from each other and from non-individually addressable locations. In some cases, the individually addressable locations may not be defined by physical features of the substrate, and instead may be defined digitally (e.g., by indexing) and/or via the analytes and/or reagents that are loaded on the substrate (e.g., the locations in which analytes are immobilized on the substrate). The plurality of individually addressable locations may be arranged as an array, randomly, or according to any pattern, on the substrate. FIG. 2 illustrates different substrates (from a top view) comprising different arrangements of individually addressable locations 201, with panel A showing a substantially rectangular substrate with regular linear arrays, panel B showing a substantially circular substrate with regular linear arrays, and panel C showing an arbitrarily shaped substrate with irregular arrays.

The substrate may have any number of individually addressable locations, for example, on the order of 1, 10¹, 10², 10³, 10⁴, 10⁵, 10⁶, 10⁷, 10⁸, 10⁹, 10¹⁰, 10¹¹, 10¹², 10¹³ or more individually addressable locations. Each individually addressable location may have any shape or form, for example the general shape or form of a circle, oval, square, rectangle, polygonal, or non-polygonal shape when viewed from the top. A plurality of individually addressable locations can have uniform shape or form, or different shapes or forms. An individually addressable location may have any size. In some cases, an individually addressable location may have an area of at least and/or at most about 0.1, 0.2, 0.25, 0.3, 0.4, 0.5, 0.6, 0.7, 0.8, 0.9, 1, 1.1, 1.2, 1.25, 1.3, 1.4, 1.5 , 1.6, 1.7, 1.75, 1.8, 1.9, 2, 2.25, 2.5, 2.75, 3, 3.25, 3.5, 3.75, 4, 4.25, 4.5, 4.75, 5, 5.5, 6, 7, 8, 9, 10 square micron (μm²), or more. The individually addressable locations may be distributed on a substrate with a pitch determined by the distance between the center of a first location and the center of the closest or neighboring individually addressable location. Locations may be spaced with a pitch of at least and/or at most about 0.1, 0.2, 0.25, 0.3, 0.4, 0.5, 0.6, 0.7, 0.8, 0.9, 1, 1.1, 1.2, 1.25, 1.3, 1.4, 1.5 , 1.6, 1.7, 1.75, 1.8, 1.9, 2, 2.25, 2.5, 2.75, 3, 3.25, 3.5, 3.75, 4, 4.25, 4.5, 4.75, 5, 5.5, 6, 6.5, 7, 7.5, 8, 8.5, 9, 9.5, or 10 micron (μm). In some cases, the pitch between two individually addressable locations may be determined as a function of a size of a loading object (e.g., bead). For example, where the loading object is a bead having a maximum diameter, the pitch may be at least about the maximum diameter of the loading object.

Each of the plurality of individually addressable locations, or each of a subset of the locations, may be capable of immobilizing thereto an analyte (e.g., a nucleic acid, a protein, a carbohydrate, etc.) or a reagent (e.g., a nucleic acid, a probe molecule, a barcode molecule, an antibody molecule, a primer molecule, a bead, etc.). In some cases, an analyte or reagent may be immobilized to an individually addressable location via a support, such as a bead. In an example, a first bead comprising a first colony of nucleic acid molecules each comprising a first template sequence is immobilized to a first individually addressable location, and a second bead comprising a second colony of nucleic acid molecules each comprising a second template sequence is immobilized to a second individually addressable location. A substrate may comprise more than one type of individually addressable location arranged as an array, randomly, or according to any pattern, on the substrate. In some cases, different types of individually addressable locations may have different chemical, physical, and/or biological properties (e.g., hydrophobicity, charge, color, topography, size, dimensions, geometry, etc.).

In some cases, an individually addressable location may comprise a distinct surface chemistry. The distinct surface chemistry may distinguish between different addressable locations and/or distinguish an individually addressable location from surrounding locations. For example, a first location type may comprise a first surface chemistry, and a second location type may lack the first surface chemistry. In another example, the first location type may comprise the first surface chemistry and the second location type may comprise a second, different surface chemistry. A first location type may have a first affinity towards an object (e.g., a bead comprising nucleic acid molecules, e.g., amplicons, immobilized thereto) and a second location type may have a second, different affinity towards the same object. In other examples, a first location type comprising a first surface chemistry may have an affinity towards a first sample type (e.g., a bead comprising nucleic acid molecules, e.g., amplicons, immobilized thereto) and exclude a second sample type (e.g., a bead lacking nucleic acid molecules, e.g., amplicons, immobilized thereto). The first location type and the second location type may or may not be disposed on the surface in alternating fashion. A first location type or region type may comprise a positively charged surface chemistry and a second location type or region type may comprise a negatively charged surface chemistry. A first location type or region type may comprise a hydrophobic surface chemistry and a second location type or region type may comprise a hydrophilic surface chemistry. A first location type comprises a binder, as described elsewhere herein, and a second location type does not comprise the binder or comprises a different binder. In some cases, a surface chemistry may comprise an amine. In some cases, a surface chemistry may comprise a silane (e.g., tetramethylsilane). In some cases, the surface chemistry may comprise hexamethyldisilazane (HMDS). In some cases, the surface chemistry may comprise (3-aminopropyl)triethoxysilane (APTMS). In some cases, the surface chemistry may comprise a surface primer molecule or any oligonucleotide molecule that has any degree of affinity towards another molecule. In one example, the substrate comprises a plurality of individually addressable locations, each defined by APTMS, which are positively charged and has affinity towards an amplified bead (e.g., a bead comprising nucleic acid molecules, e.g., amplicons, immobilized thereto) which exhibits a negative charge. The locations surrounding the plurality of individually addressable locations may comprise HMDS which repels amplified beads.

In some cases, the individually addressable locations may be indexed, e.g., spatially. Data corresponding to an indexed location, collected over multiple periods of time, may be linked to the same indexed location. In some cases, sequencing signal data collected from an indexed location, during iterations of sequencing-by-synthesis flows, are linked to the indexed location to generate a sequencing read for an analyte immobilized at the indexed location. In some embodiments, the individually addressable locations are indexed by demarcating part of the surface, such as by etching or notching the surface, using a dye or ink, depositing a topographical mark, depositing a sample (e.g., a control nucleic acid sample), depositing a reference object (e.g., e.g., a reference bead that always emits a detectable signal during detection), and the like, and the individually addressable locations may be indexed with reference to such demarcations. A combination of positive demarcations and negative demarcations (lack thereof) may be used to index the individually addressable locations. In some embodiments, each of the individually addressable locations is indexed. In some embodiments, a subset of the individually addressable locations is indexed. In some embodiments, the individually addressable locations are not indexed, and a different region of the substrate is indexed.

The substrate may comprise a planar or substantially planar surface. Substantially planar may refer to planarity at a micrometer level (e.g., a range of unevenness on the planar surface does not exceed the micrometer scale) or nanometer level (e.g., a range of unevenness on the planar surface does not exceed the nanometer scale). Alternatively, substantially planar may refer to planarity at less than a nanometer level or greater than a micrometer level (e.g., millimeter level). Alternatively or in addition, a surface of the substrate may be textured or patterned. For example, the substrate may comprise grooves, troughs, hills, pillars, wells, cavities (e.g., micro-scale cavities or nano-scale cavities), and/or channels. The substrate may have regular textures and/or patterns across the surface of the substrate. The substrate may have regular geometric structures (e.g., wedges, cuboids, cylinders, spheroids, hemispheres, etc.) above or below a reference level of the surface. Alternatively, the substrate may have irregular textures and/or patterns across the surface of the substrate. The substrate may be textured or patterned such that all features are at or above a reference level of the surface (no features below a reference level of the surface, such as a well). The substrate may be textured or patterned such that all features are at or below a reference level of the surface (no features below a reference level of the surface, such as a pillar). In some instances, a texture of the substrate may comprise structures having a maximum dimension of at most about 500%, 400%, 300%, 200%, 100%, 90%, 80%, 70%, 60%, 50%, 40%, 30%, 20%, 10%, 9%, 8%, 7%, 6%, 5%, 4%, 3%, 2%, 1%, 0.1%, 0.01%, 0.001%, 0.0001%, 0.00001% of the total thickness of the substrate or a layer of the substrate. In some instances, the textures and/or patterns of the substrate may define at least part of an individually addressable location on the substrate. A textured and/or patterned substrate may be substantially planar. Alternatively, the substrate may be untextured and unpatterned.

A binder may be configured to immobilize an analyte or reagent to an individually addressable location. In some cases, a surface chemistry of an individually addressable location may comprise one or more binders. In some cases, a plurality of individually addressable locations may be coated with binders. The binders may be integral to the substrate. The binders may be added to the substrate. For instance, the binders may be added to the substrate as one or more coating layers. The substrate may comprise an order of magnitude of at least and/or at most about 10, 10², 10³, 10⁴, 10⁵, 10⁶, 10⁷, 10⁸, 10⁹, 10¹⁰, 10¹¹, 10¹², 10¹³ or more binders. The binders may immobilize analytes or reagents through non-specific interactions, such as one or more of hydrophilic interactions, hydrophobic interactions, electrostatic interactions, physical interactions (for instance, adhesion to pillars or settling within wells), and the like. Alternatively or in addition, the binders may immobilize analytes or reagents through specific interactions. For instance, where the analyte or reagent is a nucleic acid molecule, the binders may comprise oligonucleotide adaptors configured to bind to the nucleic acid molecule. In other examples, the binders may comprise one or more of antibodies, oligonucleotides, nucleic acid molecules, aptamers, affinity binding proteins, lipids, carbohydrates, and the like. The binders may immobilize analytes or reagents through any possible combination of interactions. For instance, the binders may immobilize nucleic acid molecules through a combination of physical and chemical interactions, through a combination of protein and nucleic acid interactions, etc. A single binder may bind a single analyte or single reagent, a single binder may bind a plurality of analytes or a plurality of reagents, or a plurality of binders may bind a single analyte or a single reagent. In some instances, the substrate may comprise a plurality of types of binders, for example to bind different types of analytes or reagents. For example, a first type of binders (e.g., oligonucleotides) are configured to bind a first type of analyte (e.g., nucleic acid molecules) or reagent, and a second type of binders (e.g., antibodies) are configured to bind a second type of analyte (e.g., proteins) or reagent. In another example, a first type of binders (e.g., first type of oligonucleotide molecules) are configured to bind a first type of nucleic acid molecules and a second type of binders (e.g., second type of oligonucleotide molecules) are configured to bind a second type of nucleic acid molecules. For example, the substrate may be configured to bind different types of analytes or reagents in certain fractions or specific locations on the substrate by having the different types of binders in the certain fractions or specific locations on the substrate.

The substrate may be rotatable about an axis, referred to herein as a rotational axis. The rotational axis may or may not be an axis through the center of the substrate. The systems, devices, and apparatus described herein may further comprise an automated or manual rotational unit configured to rotate the substrate. The rotational unit may comprise a motor and/or a rotor. For instance, the substrate may be affixed to a chuck (such as a vacuum chuck). The substrate may be rotated at a rotational speed of at least about 1 revolution per minute (rpm), at least 2 rpm, at least 5 rpm, at least 10 rpm, at least 20 rpm, at least 50 rpm, at least 100 rpm, at least 200 rpm, at least 500 rpm, at least 1,000 rpm, at least 2,000 rpm, at least 5,000 rpm, at least 10,000 rpm, or greater. Alternatively or in addition, the substrate may be rotated at a rotational speed of at most about 10,000 rpm, 5,000 rpm, 2,000 rpm, 1,000 rpm, 500 rpm, 200 rpm, 100 rpm, 50 rpm, 20 rpm, 10 rpm, 5 rpm, 2 rpm, 1 rpm, or less. The substrate may be configured to rotate with different rotational velocities during different operations described herein, for example with higher velocities during reagent dispense and with lower velocities during analyte loading and imaging operations. The substrate may be configured to rotate with a rotational velocity that varies according to a time-dependent function, such as a ramp, sinusoid, pulse, or other function or combination of functions. The time-varying function may be periodic or aperiodic.

Analytes or reagents may be immobilized to the substrate during rotation. Analytes or reagents may be dispensed onto the substrate prior to or during rotation of the substrate. When the substrate is rotated at a relatively high rotational velocity, high speed coating across the substrate may be achieved via tangential inertia directing unconstrained spinning reagents in a partially radial direction (that is, away from the axis of rotation) during rotation, a phenomenon commonly referred to as centrifugal force. In some cases, the substrate may be rotated at relatively low velocities such that reagents dispensed to a certain location do not move to another location, or moves minimally, because of the rotation, to permit controlled dispensing of reagents to desired locations. For example, bead loading may be performed with controlled dispensing. For controlled dispensing, the substrate may be rotating with a rotational frequency of no more than 60, 50, 40, 30, 25, 20 15, 14, 13, 12, 11, 10, 9, 8, 7, 6, 5, 4, 3, 2, 1 rpm or less. In some cases the substrate may be rotating with a rotational frequency of about 5 rpm during controlled dispensing. A speed of substrate rotation may be adjusted according to the appropriate operation (e.g., high speed for spin-coating, high speed for washing the substrate, low speed for sample loading, low speed for detection, low speed for analyte or reagent incubation, etc.).

In some cases, the substrate may be movable in any vector or direction. For example, such motion may be non-linear (e.g., in rotation about an axis), linear (e.g., on a rail track), or a hybrid of linear and non-linear motion. In some instances, the systems, devices, and apparatus described herein may further comprise a motion unit configured to move the substrate. The motion unit may comprise any mechanical component, such as a motor, rotor, actuator, linear stage, drum, roller, pulleys, etc., to move the substrate. Analytes or reagents may be immobilized to the substrate during any such motion. Analytes or reagents may be dispensed onto the substrate prior to, during, or subsequent to motion of the substrate.

### Loading Reagents Onto an Open Substrate

The surface of the substrate may be in fluid communication with at least one fluid nozzle (of a fluid channel). The surface may be in fluid communication with the fluid nozzle via a non-solid gap, e.g., an air gap. In some cases, the surface may additionally be in fluid communication with at least one fluid outlet. The surface may be in fluid communication with the fluid outlet via an air gap. The nozzle may be configured to direct a solution to the array. The outlet may be configured to receive a solution from the substrate surface. The solution may be directed to the surface using one or more dispensing nozzles. For example, the solution may be directed to the array using at least 1, 2, 3, 4, 5, 6, 7, 8, 9, 10, 11, 12, 13, 14, 15, 16, 17, 18, 19, 20 or more dispensing nozzles. In some cases, different reagents (e.g., nucleotide solutions of different types, different probes, washing solutions, etc.) may be dispensed via different nozzles, such as to prevent contamination. Each nozzle may be connected to a dedicated fluidic line or fluidic valve, which may further prevent contamination. Alternatively, some nozzles may share a fluidic line or fluidic valve, such as for pre-dispense mixing and/or to dispensing to multiple locations. A type of reagent may be dispensed via one or more nozzles. The one or more nozzles may be directed at or in proximity to a center of the substrate. Alternatively, the one or more nozzles may be directed at or in proximity to a location on the substrate other than the center of the substrate. Alternatively or in combination, one or more nozzles may be directed closer to the center of the substrate than one or more of the other nozzles. For instance, one or more nozzles used for dispensing washing reagents may be directed closer to the center of the substrate than one or more nozzles used for dispensing active reagents. The one or more nozzles may be arranged at different radii from the center of the substrate. The nozzles may be angled towards or away from a center of the substrate, or not angled (e.g., normal to the substrate plane). Two or more nozzles may be operated in combination to deliver fluids to the substrate more efficiently. One or more nozzles may be configured to deliver fluids to the substrate as a jet, spray (or other dispersed fluid), and/or droplets. One or more nozzles may be operated to nebulize fluids prior to delivery to the substrate. For example, the fluids may be delivered as aerosol particles.

In some cases, the solution may be dispensed on the substrate while the substrate is stationary; the substrate may then be subjected to rotation (or other motion) following the dispensing of the solution. Alternatively, the substrate may be subjected to rotation (or other motion) prior to the dispensing of the solution; the solution may then be dispensed on the substrate while the substrate is rotating (or otherwise moving). In some cases, rotation of the substrate may yield a centrifugal force (or inertial force directed away from the axis) on the solution, causing the solution to flow radially outward over the array. In this manner, rotation of the substrate may direct the solution across the array. Continued rotation of the substrate over a period of time may dispense a fluid film of a nearly constant thickness across the array.

One or more conditions such as the rotational velocity of the substrate, the acceleration of the substrate (e.g., the rate of change of velocity), viscosity of the solution, angle of dispensing (e.g., contact angle of a stream of reagents) of the solution, radial coordinates of dispensing of the solution (e.g., on center, off center, etc.), temperature of the substrate, temperature of the solution, and other factors may be adjusted and/or otherwise optimized to attain a desired wetting on the substrate and/or a film thickness on the substrate, such as to facilitate uniform coating of the substrate. For instance, one or more conditions may be applied to attain a film thickness of at least and/or at most about 10 nanometers (nm), 20 nm, 50 nm, 100 nm, 200 nm, 500 nm, 1 micrometer (μm), 2 μm, 5 μm, 10 μm, 20 μm, 50 μm, 100 μm, 200 μm, 500 μm, 1 millimeter (mm), or more. One or more conditions may be applied to attain a film thickness that is within a range defined by any two of the preceding values. In some cases, a surfactant may be added to the solution, or a surfactant may be added to the surface to facilitate uniform coating or to facilitate sample loading efficiency. Alternatively or in conjunction, the thickness of the solution may be adjusted using mechanical, electric, physical, or other mechanisms. For example, the solution may be dispensed onto a substrate and subsequently leveled using, e.g., a physical scraper such as a squeegee, to obtain a desired thickness of uniformity across the substrate.

Reagents may be dispensed to the substrate to multiple locations, and/or multiple reagents may be dispensed to the substrate to a single location, via different mechanisms. Reagent dispensing mechanisms disclosed herein may be applicable to sample dispensing. For example, a reagent may comprise the sample. The term “loading onto a substrate,” as used herein, may refer to dispensing of the reagent or the sample to a surface of the substrate in accordance with any reagent dispensing mechanism described herein.

In some cases, dispensing may be achieved via relative motion of the substrate and the dispenser (e.g., nozzle). For example, a reagent may be dispensed to the substrate at a first location, and thereafter travel to a second location different from the first location due to forces (e.g., centrifugal forces, centripetal forces, inertial forces, etc.) caused by motion of the substrate (e.g., rotational motion of the substrate, linear motion of the substrate, combination thereof, etc.). In another example, a reagent may be dispensed to a reference location, and the substrate may be moved relative to the reference location such that the reagent is dispensed to multiple locations of the substrate. In another example, a dispenser may be moved relative to the substrate to dispense the reagent at different locations, for example moved prior to, during, or subsequent to dispensing. In an example, a reagent is ‘painted’ onto the substrate by moving the dispenser and/or the substrate relative to each other, along a desired path on the substrate. The open substrate geometry may allow for flexible and controlled dispensing of a reagent to a desired location on the substrate. In some cases, dispensing may be achieved without relative motion between the substrate and the dispenser. For example, multiple dispensers may be used to dispense reagents to different locations, and/or multiple reagents to a single location, or a combination thereof (e.g., multiple reagents to multiple locations).

In another example, an external force (e.g., involving a pressure differential, involving physical force, involving a magnetic force, involving an electrical force, etc.), such as wind, a field-generating device, or a physical device, may be applied to one or more surfaces of the substrate to direct reagents to different locations across the substrate. In another example, the method for dispensing reagents may comprise vibration. In such an example, reagents may be distributed or dispensed onto a single region or multiple regions of the substrate. The substrate may then be subjected to vibration, which may spread the reagent to different locations across the substrate. Alternatively or in conjunction, the method may comprise using mechanical, electric, physical, or other mechanisms to dispense reagents to the substrate. For example, the solution may be dispensed onto a substrate and a physical scraper (e.g., a squeegee) may be used to spread the dispensed material or spread the reagents to different locations and/or to obtain a desired thickness or uniformity across the substrate. Beneficially, such flexible dispensing may be achieved without contamination of the reagents.

In some instances, where a volume of reagent is dispensed to the substrate at a first location, and thereafter travels to a second location different from the first location, the volume of reagent may travel in a path or paths, such that the travel path or paths are coated with the reagent. In some cases, such travel path or paths may encompass a desired surface area (e.g., entire surface area, partial surface area(s), etc.) of the substrate. In some instances, two or more reagents may be mixed on the surface of the substrate, such as by being dispensed at the same location and/or by directing a first reagent to travel to meet additional reagent(s). In some instances, the mixture of reagents formed on the substrate may be homogenous or substantially homogenous. The mixture of reagents may be formed at a first location on the substrate prior to dispersing the mixing of reagents to other locations on the substrate, such as at locations to meet other reagents or analytes.

In some embodiments, one or more solutions may be delivered directly to the reaction site without substantial displacement of the one or more solution from the point of delivery. Methods of direct delivery of a solution to the reaction site may include aerosol delivery of the solution, applying the solution using an applicator, curtain-coating the solution, slot-die coating, dispensing the solution from a translating dispense probe, dispensing the solution from an array of dispense probes, dipping the substrate into the solution, or contacting the substrate to a sheet comprising the solution.

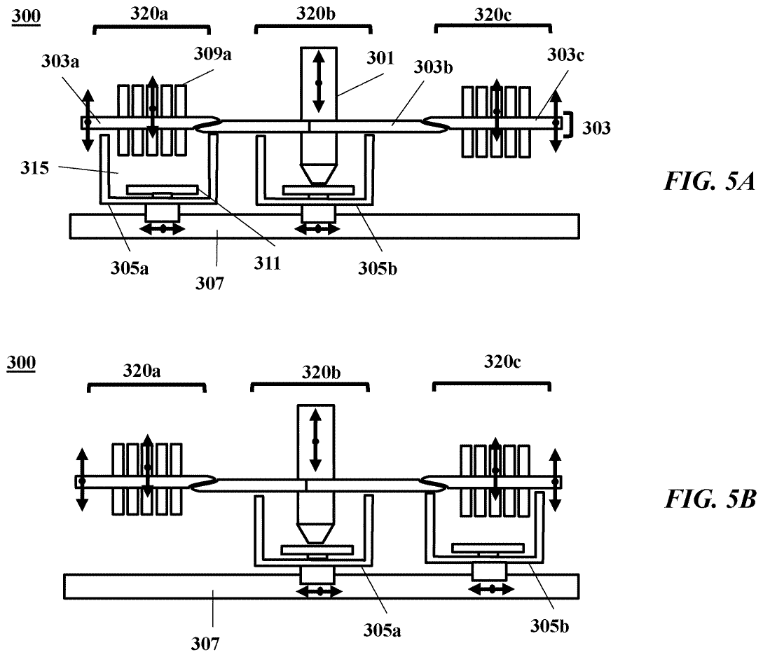

Aerosol delivery may comprise delivering a solution to the substrate in aerosol form by directing the solution to the substrate using a pressure nozzle or an ultrasonic nozzle. Applying the solution using an applicator may comprise contacting the substrate with an applicator comprising the solution and translating the applicator relative to the substrate. For example, applying the solution using an applicator may comprise painting the substrate. The solution may be applied in a pattern by translating the applicator, rotating the substrate, translating the substrate, or a combination thereof. Curtain-coating may comprise dispensing the solution from a dispense probe to the substrate in a continuous stream (e.g., a curtain or a flat sheet) and translating the dispense probe relative to the substrate. A solution may be curtain-coated in a pattern by translating the dispense probe, rotating the substrate, translating the substrate, or a combination thereof. Slot-die coating may comprise dispensing the solution from a dispense probe positioned near the substrate such that the solution forms a meniscus between the substrate and the dispense probe and translating the dispense probe relative to the substrate. A solution may be slot-die coated in a pattern by translating the dispense probe, rotating the substrate, translating the substrate, or a combination thereof. Dispensing the solution from a translating dispense probe may comprise translating the dispense probe relative to the substrate in a pattern (e.g., a spiral pattern, a circular pattern, a linear pattern, a striped pattern, a cross-hatched pattern, or a diagonal pattern). Dispensing the solution from an array of dispense probes may comprise dispensing the solution from an array of nozzles (e.g., a shower head) positioned above the substrate such that the solution is dispensed across an area of the substrate substantially simultaneously. Dipping the substrate into the solution may comprise dipping the substrate into a reservoir comprising the solution. In some embodiments, the reservoir may be a shallow reservoir to reduce the volume of the solution required to coat the substrate. Contacting the substrate to a sheet comprising the solution may comprise bringing the substrate in contact with a sheet of material (e.g., a porous sheet or a fibrous sheet) permeated with the solution. The solution may be transferred to the substrate. In some embodiments, the sheet of material may be a single-use sheet. In some embodiments, the sheet of material may be a reusable sheet. In some embodiments, a solution may be dispensed onto a substrate using the method illustrated in FIG. 5B, where a jet of a solution may be dispensed from a nozzle to a rotating substrate. The nozzle may translate radially relative to the rotating substrate, thereby dispensing the solution in a spiral pattern onto the substrate.

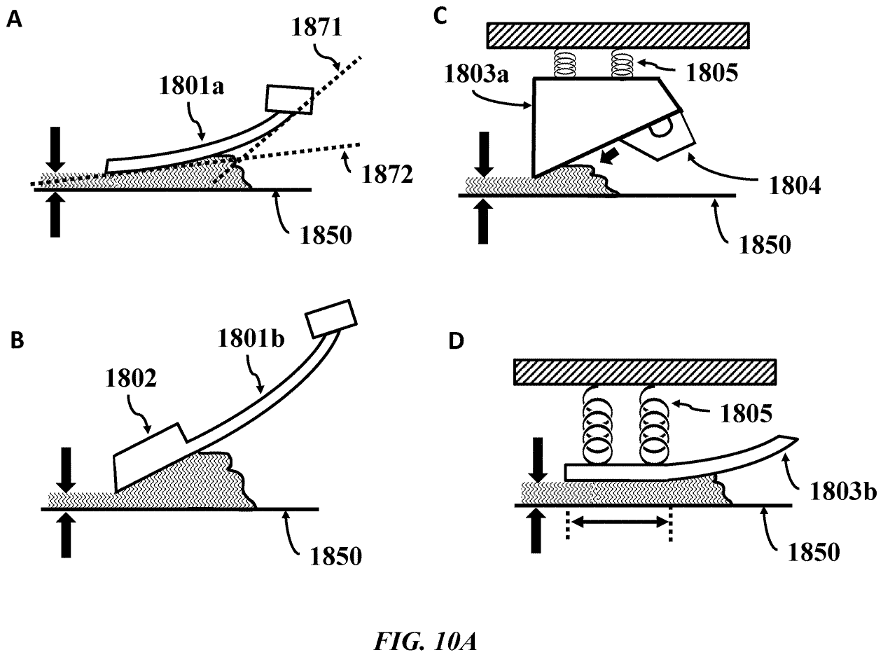

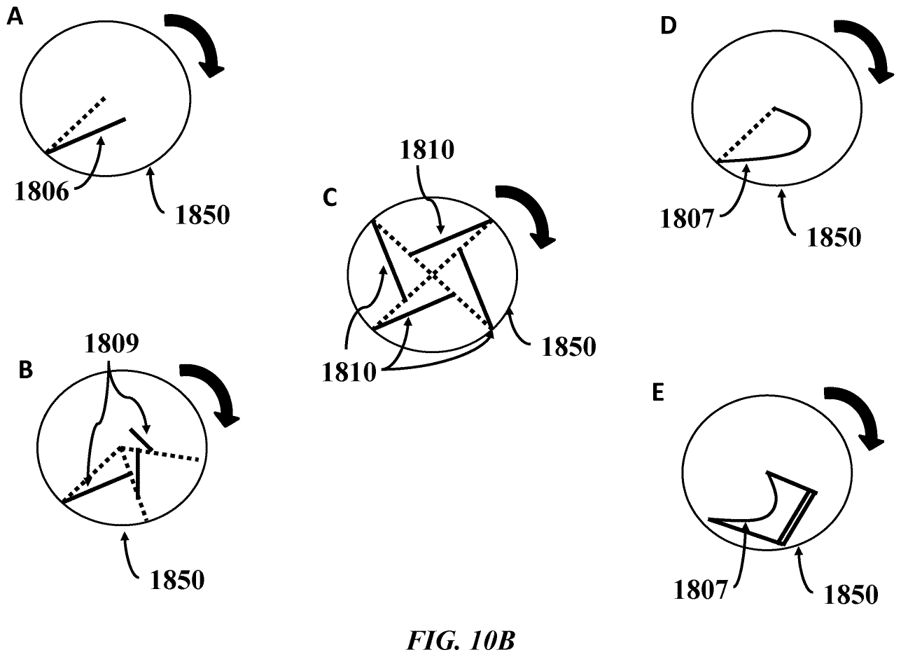

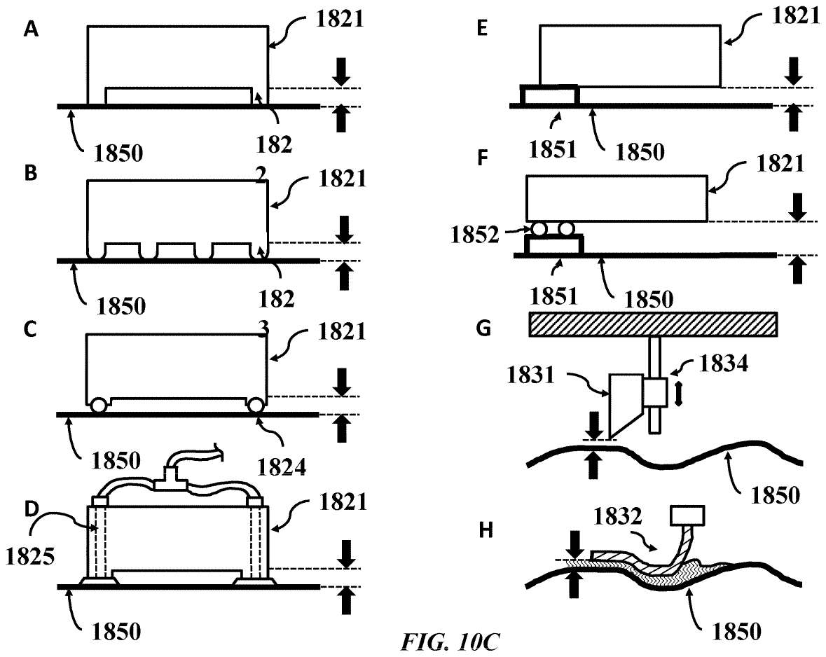

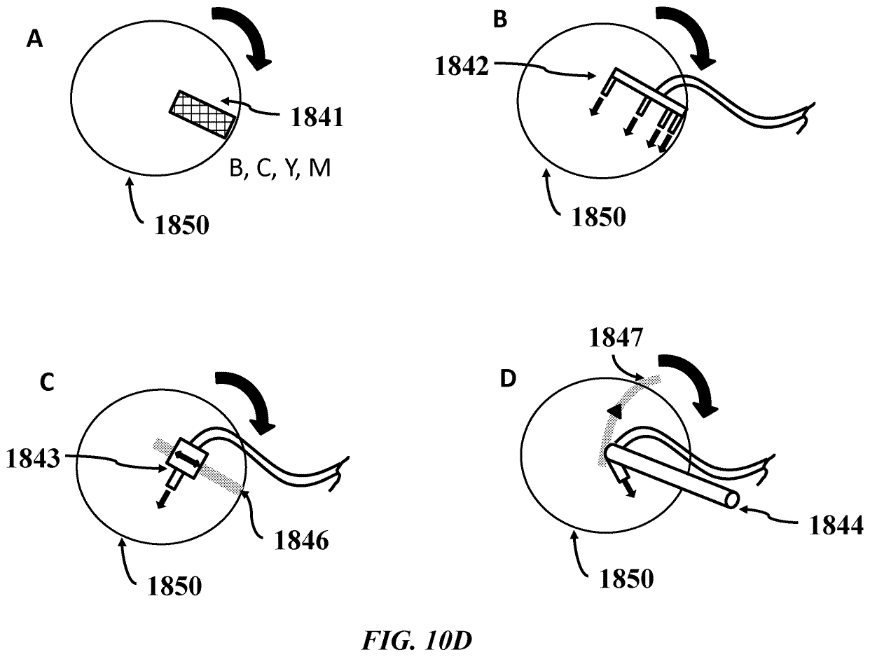

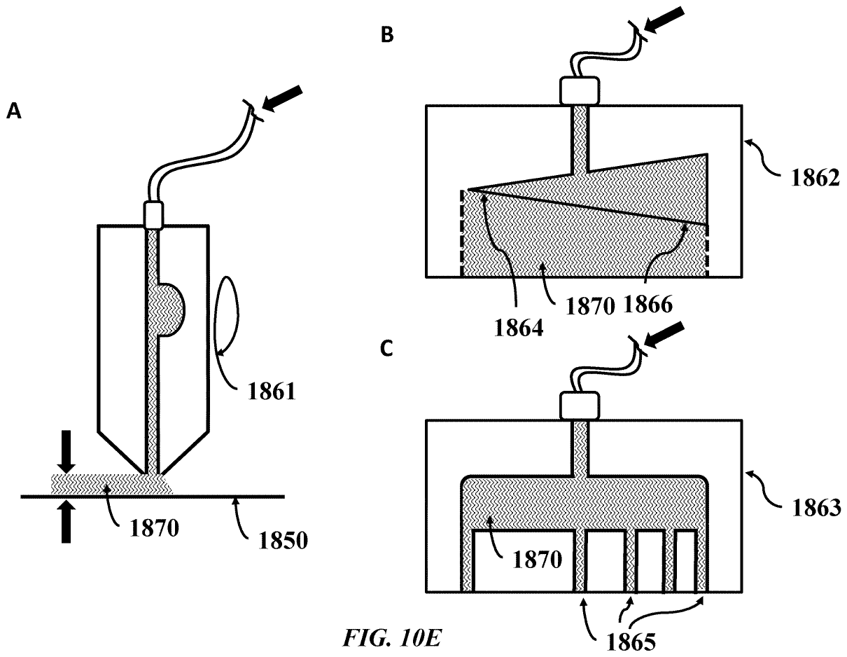

FIGS. 10A-10E illustrate multiple dispense mechanisms that can be used with the systems described herein, such as to dispense and/or disperse reagents and/or samples to a substrate. FIGS. 10A-10C illustrate different schematics of using a physical scraper, such as a doctor blade tool, a mop tool, or a rigid object tool, to disperse reagents across different regions of a substrate. FIG. 10D illustrate different schematics of using inkjet heads, multi-needle dispensers, single-needle dispensers, and actuated dispensers. FIG. 10E illustrate different schematics of using slot die tools to dispense fluids to a substrate.

A physical scraper may be used to disperse fluids, such as reagents or samples, across different regions of a substrate via relative motion between the physical scraper and the substrate. The physical scraper may be a rigid object or a flexible, deformable object. Some non-limiting examples of physical scrapers include a squeegee tool, a doctor blade tool, a mop tool, other flexible or deformable tool, or a rigid object tool. In some cases, the physical scraper may contact the substrate surface as it travels from one region relative to another region of the substrate. In some cases, the physical scraper may not contact the substrate surface, in one example by maintaining a minimum distance from the substrate surface, as it relatively travels from one region to another region on the substrate. Beneficially, this may prevent displacement of previously immobilized objects on the substrate surface (e.g., beads comprising nucleic acid molecules) due to the action of the physical scraper. In some cases, even when the physical scraper is not contacting the substrate surface, the physical scraper may contact the fluid on the substrate surface as it relatively travels from one region to another region of the substrate. In some cases, the physical scraper may maintain or leave behind a fluid film on the substrate surface. In some cases, the physical scraper may alternate between contacting the substrate and not contacting the substrate as it relatively moves from one region to another region on the substrate. In some cases, the physical scraper may alternate between contacting the fluid and not contacting the fluid on the substrate surface as it relatively moves from one region to another region on the substrate. It will be appreciated that the physical scraper may be stationary, and the substrate may move relative to the physical scraper; the substrate may be stationary, and the physical scraper may move relative to the substrate; or both the physical scraper and the substrate may be in active motion relative to the other.

In FIG. 10A, panel A illustrates a cross-sectional side view of a single, flexible blade 1801a dispersing fluid across a substrate 1850. The blade 1801a may have a deflection angle 1871 and contact angle 1872 relative to the substrate surface. Such angles may be optimized for efficient fluid dispersal. The blade 1801a may be set to not contact the substrate surface, such as to leave behind a fluid film (e.g., reagent film, sample film, etc.) on the substrate surface along its travel path. The flexibility of the blade may allow the blade to bend in either direction as it relatively travels across the substrate surface to account for non-uniformities on the substrate surface, such as height deviations. Panel B illustrates a cross-sectional side view of a single, flexible blade 1801b which contains a contact end 1802 of different design than that shown in Panel A. The blade 1801b may be set to not contact the substrate surface, such as to leave behind a fluid film (e.g., reagent film, sample film, etc.) on the substrate surface along its travel path. In some cases, not shown in panels A-B, the blade end proximal to the substrate surface may comprise one or more standoffs or spacers (described in more detailed with respect to FIG. 10C), which may or may not contact the substrate surface, to set a height of the remaining blade portion from the substrate surface. In such cases, a stiffer or more rigid blade may reduce the bow between the standoffs or spacers. Panel C illustrates a cross-sectional side view of a single blade 1803a dispersing fluid across a substrate 1850, which blade 1803a is held above the substrate surface via one or more springs 1805. The blade 1803a may be flexible or rigid. In either case, the one or more springs 1805 may provide flexibility in height movement of the blade, such as to account for non-uniformities on the substrate surface. In some cases, the blade 1803a may further comprise a slot die 1804 or other nozzle (or nozzles) configured to dispense fluid, such as wash fluid, directly onto or along the blade 1801a. The slot die or other nozzle may be integrated into the blade 1801a or may be an independent system coupled to the blade 1801a. Beneficially, the blade may be washed in between different flows to prevent contamination via the blade carrying over fluids (e.g., reagents, samples) from previous flows. The same wash buffer washing the blade may be used downstream to wash the substrate between the flows. Panel D illustrates a cross-sectional side view of a single blade 1803b dispersing fluid across a substrate 1850, which blade 1803b is held above the substrate surface via one or more springs 1805. The blade 1803b may be flexible or rigid. In either case, the one or more springs 1805 may provide flexibility in height movement of the blade, such as to account for non-uniformities on the substrate surface. In some cases, the blade may have a relatively long horizontal contact portion with the fluid on the substrate surface, such that the contact angle of the blade is closer to 0. The larger horizontal contact area may facilitate better tracking of the blade along the surface profile, similar to slide film damping.

In FIG. 10B, panel A illustrates a top view of a single blade 1806 dispersing fluid across a rotating substrate 1850. The blade 1806 may be oriented at a predetermined chord angle to facilitate faster, radially outward movement of the fluid across the substrate. Panels B and C each illustrate a top view of multiple blades 1809 and multiple blades 1810, respectively, dispersing fluid across a rotating substrate 1850. The blades 1809 and 1810 may each be oriented at a predetermined chord angle to facilitate faster, radially outward movement of the fluid across the substrate. The multiple blades may improve coverage and spreading of the fluid. Panel B shows multiple blades having different blade lengths, and panel C shows multiple blades having uniform blade lengths. Any number of blades may be used to optimize coverage and fluid dispersal efficiency. Any arrangement of the blade(s) may be used to optimize coverage and fluid dispersal efficiency. Panel D illustrates a top view of a single, curved blade 1807 dispersing fluid across a rotating substrate 1850. The curved blade 1807 may expand across the full radius to cover the full area of the substrate during rotational movement, but also enable the angles to facilitate faster, radially outward movement of the fluid across the substrate. In some cases, the curvature of the blade 1807 may be maintained via rigidity of the blade, where flexibility across blade height movement from the surface may be provided by springs (e.g., 1805) or other mechanisms (e.g., see FIG. 10C, panel G). Panel E illustrates a projection of the curved blade 1807 of Panel D.

In FIG. 10C, Panel A illustrates a cross-sectional side view of a blade 1821 with two spacers 1822 disposed at a first end of the blade 1821, the first end being proximal to the substrate 1850 surface. A spacer surface or edge facing the substrate surface may be closer to the substrate surface (sometimes contacting the substrate surface) than a non-spacer blade surface or edge facing the substrate surface (which is not contacting the substrate surface). The spacers may help set the blade height from the surface. In some cases, the spacers may be integrated with the blade material. In some cases, the spacers may be composed of alternate material from the blade material, for example coupled to the blade. The spacer material may be selected to help reduce friction and/or improve wear. The blade may comprise any number of spacers, such as 1, 2, 3, 4, 5, 6, 7, 8, 9, 10 or more spacers, arranged in any design (e.g., in a regular or irregular array, or ordered or random pattern, etc.) at the first end of the blade. As an example, Panel A illustrates the two spacers 1822 as being disposed at opposing ends of the first end of the blade. Panel B illustrates a cross-sectional side view of a blade 1821 with four spacers 1823 disposed at a first end of the blade 1821, the first end being proximal to the substrate 1850 surface, and the four spacers 1823 being substantially evenly spaced apart across the first end of the blade.

Panel C illustrates a cross-sectional side view of two rollers, spherical bearings, or ball bearings 1824 disposed at a first end of the blade 1821, the first end being proximal to the substrate 1850 surface. A roller, spherical bearing, or ball bearing may contact and roll and/or slide across the substrate surface while maintaining a non-roller blade surface or edge at a distance from the substrate surface. The roller, spherical bearing, or ball bearing may help set the blade height from the surface. The roller material may be selected to help reduce friction and/or improve wear. The blade may comprise any number of rollers, spherical bearings, or ball bearings such as 1, 2, 3, 4, 5, 6, 7, 8, 9, 10 or more, arranged in any design (e.g., in a regular or irregular array, or ordered or random pattern, etc.) at the first end of the blade. As an example, Panel C illustrates the two rollers, spherical bearings, or ball bearings 1824 as being disposed at opposing ends of the first end of the blade. Panel D illustrates a cross-sectional side view of a blade 1821 comprising two fluid bearings 1825 in which a fluid (e.g., liquid or gas), such as water, oil, or air, can be pressurized between a blade 1821 surface and a substrate 1850 surface. Panel E illustrates a cross-sectional side view of a substrate 1850 comprising a sticker or other bearing surface 1851, which is elevated from non-bearing surfaces of the substrate, the sticker or other bearing surface 1851 interfacing a blade 1821. The sticker or other bearing surface 1851 may be closer to the blade surface or edge facing the substrate surface than the non-bearing surfaces of the substrate. The sticker or other bearing surface may help set the blade height from the substrate surface. In some cases, the sticker or other bearing surface may be integrated with the substrate material. In some cases, the sticker or other bearing surface may be composed of alternate material from the substrate material, for example coupled to the substrate. The sticker or other bearing surface material may be selected to help reduce friction and/or improve wear. The sticker or other bearing surface material may experience different levels of wear across radial distances from the rotational axis of the substrate as the substrate rotates and interfaces the blade, where the bearing portions closer to the rotational axis experiences less wear due to lower tangential velocity compared to the bearing portions farther from the rotational axis—in some cases, the bearing material may be designed to equalize wear across different radial positions of the bearing material. The substrate may comprise any number of stickers or other bearing surfaces, such as 1, 2, 3, 4, 5, 6, 7, 8, 9, 10 or more, arranged in any design (e.g., in a regular or irregular array, or ordered or random pattern, etc.) and configured to interact with the blade 1821. Panel F illustrates a cross-sectional side view of the design of Panel E with additional rollers, spherical bearings, or ball bearings 1852 disposed above the sticker or other bearing surface 1851 of the substrate 1850. The roller, spherical bearing, or ball bearing 1852 on the sticker or other bearing surface 1851 may contact and roll and/or slide across the blade surface or edge while maintaining the blade surface or edge at a distance from the substrate surface. The roller material may be selected to help reduce friction and/or improve wear. The sticker or other bearing surface may comprise any number of rollers, spherical bearings, or ball bearings such as 1, 2, 3, 4, 5, 6, 7, 8, 9, 10 or more, arranged in any design (e.g., in a regular or irregular array, or ordered or random pattern, etc.) above the sticker or other bearing surface. In some cases, the number and location of rollers, spherical bearings, or ball bearings may be selected to better control or optimize the angle of the blade relative to the substrate surface.

Panel G illustrates a cross-sectional side view of an active blade height control system 1834 comprising a blade 1831 which is configured to move along a z axis (substantially perpendicular to a plane of a surface of the substrate 1850 or substantially parallel to a direction of gravitational force) relative to the surface of the substrate 1850. The blade height control system 1834, in a non-limiting example, may be informed of substrate surface profile data by one or more sensors, for example an autofocus system, and configured to move the blade 1831 in the z axis based on the data as the blade and substrate surface relatively transitions (e.g., linear movement, rotational movement, both linear and rotational movement, etc.). In some cases, a map of the substrate surface profile may be generated or learned, and the same map may be used to inform one or more actuators that are configured to move the blade height through each cycle and/or repeated cycles. In some cases, a map of the substrate surface profile may be generated, learned, and/or estimated in real-time or substantially real-time by one or more sensors while the blade and substrate are in relative motion. In some cases, the blade height control system 1834 may be configured to adjust a height of the blade within at most about 100 μm, 90 μm, 80 μm, 70 μm, 60 μm, 50 μm, 40 μm, 30 μm, 20 μm, 19 μm, 18 μm, 17 μm, 16 μm, 15 μm, 14 μm, 13 μm, 12 μm, 11 μm, 10 μm, 9 μm, 8 μm, 7 μm, 6 μm, 5 μm or less from the surface profile.

An autofocus system may comprise an autofocus illumination source. An autofocus system may map part or all of a surface profile of the substrate to generate an autofocus map of the surface. The autofocus map of the surface may comprise surface textures, irregularities, or tilts. In one application, during detection, the autofocus map may be used to anticipate a focal position of the surface and adjust the position of the surface relative to an objective to correct for the surface textures, irregularities, or tilts. The autofocus system may be configured to send a signal to a focusing system to adjust a position of the surface relative to the objective, thereby returning the surface to a focused position relative to the detector. In another application, during reagent and/or sample dispensing or dispersing, the autofocus map may be used to anticipate surface irregularities and send a signal to one or more actuators to adjust a position (e.g., height relative to surface) of one or more dispense or dispersal tools described herein (e.g., blade, mop, slot-die, print head, etc.) to track the surface profile of the substrate. In some cases the autofocus system may map part or all of a surface prior to scanning the surface to generate an autofocus map of the surface. In some cases, the autofocus system may map a first part of the surface (e.g., a first ring) before scanning the first part of the surface. The map of the first part of the surface may be used to anticipate and adjust the focus of the surface while scanning the first part of the surface. The map of the first part of the surface may be used to predict the focus of the surface while scanning a second part of the surface (e.g., a second ring). The second portion of the surface may be close to the first part of the surface so that the map of the first part of the surface may approximate a map of the second part of the surface. The autofocus system may map the second portion of the surface while scanning the second portion of the surface. The map of the second part of the surface may be used to anticipate and adjust the focus of the surface while scanning the third part of the surface (e.g., a third ring). In some cases, a map generated while scanning a third, fourth, fifth, sixth, seventh, eighth, ninth, tenth, eleventh, twelfth, or more portion of a surface may be used to anticipate and adjust the focus of the surface while scanning a fourth, fifth, sixth, seventh, eighth, ninth, tenth, eleventh, twelfth, thirtieth, or more part of the surface, respectively. Sequential surface portions may be positioned close together such that the map of a preceding part of the surface may approximate a map of the following part of the surface. In some cases, the autofocus system may map the entire surface before scanning. In some cases, the autofocus system may adjust the focus while scanning without generating a map.

Panel H illustrates a cross-sectional side view of a mop 1832 comprising a flexible material that is configured to track the substrate surface profile while maintaining a distance from the surface. The mop may be configured to actuate along a z axis (substantially perpendicular to a plane of the surface of the substrate 1850 or substantially parallel to a direction of gravitational force) relative to the surface of the substrate 1850 as the mop 1832 and substrate surface relatively transitions (e.g., linear movement, rotational movement, both linear and rotational movement, etc.). For example, such actuation may be via spring-loaded fixations and/or one or more actuators. The flexibility of the mop 1832 may account for irregularities in the substrate surface profile (e.g., height deviations). In some cases, the hydrodynamic pressure of the fluid film on the substrate surface may keep the mop from contacting the substrate surface.

FIG. 10D illustrates in panel A a top view of an inkjet, pico-jet, or other print head (e.g., 800) that may dispense different-colored jet streams or droplets for different nucleotide types (or otherwise different reagents) onto the substrate 1850. Panel B illustrates a top view of a multi-needle dispenser 1842 that may simultaneously or successively dispense reagents and/or samples from multiple dispense nozzles onto the substrate 1850. The multiple dispense nozzles may be arranged and/or optimized in any matter. For example, the multiple dispense nozzles may be spaced such that each nozzle covers an equal area of the substrate surface with fluid. In another example, the multiple dispense nozzles may be spaced evenly, and each nozzle may have varying orifice size to vary flow rate from each nozzle such as to cover an equal area of the substrate surface with fluid. Panel C illustrates a top view of a single-needle dispenser 1843 that may be actuated along a linear track 1846 above the substrate 1850 (e.g., via a linear actuator) and dispense reagents and/or samples from a single dispense nozzle onto the substrate 1850. Panel D illustrates a top view of a single-needle dispenser 1844 that may be actuated along a curved track 1847 (e.g., via a rotary actuator) and dispense reagents and/or samples from a single dispense nozzle onto the substrate 1850. Different features from the various panels may be combined, for example to combine a multi-needle dispenser with one or more actuators along one or more linear and/or non-linear tracks, or to combine an inkjet head with one or more actuators along one or more linear and/or non-linear tracks.

While Panels B-D illustrate the needle or nozzle at a certain angle, it will be appreciated that the angle of any needle or nozzle, described herein, relative to the substrate surface may be varied and/or optimized for efficient fluid delivery across the substrate surface. In some cases, an angled dispenser may have a proximal end (closer to the substrate surface) point radially outwards relative to the substrate surface. Such angling may reduce the relative horizontal velocity between the substrate and the dispensed stream. Beneficially, the reagents and/or samples may be dispensed at relatively larger radii on a rotating substrate, with minimized splashing or bouncing or the dispensed stream, leading to reduction in fluid loss and higher efficiency fluid delivery. In some cases, the flow velocity of the dispensed fluid may be selected to match the tangential velocity of the substrate where the fluid contacts the substrate.

FIG. 10E illustrates different slot-die tools that can be used to dispense reagents and/or samples onto a substrate 1850. Panel A illustrates a cross-sectional side view of a slot-die head 1861 dispensing fluid 1870 onto the substrate 1850 surface. The slot-die head may comprise a cavity having any profile, such as a hemi-spherical side profile as illustrated in panel A. The cavity may have a linear side profile. The cavity may have a non-linear side profile. The cavity may comprise side profiles having a combination of linear and non-linear edges. The cavity may be designed to optimize pressure drop along one or more directions, for example. Panel B illustrates a cross-sectional side view of a slot-die head 1862 dispensing fluid 1870. The slot-die head may comprise a cavity having a substantially triangular or horn-shaped side profile which tapers to the left 1864 (as seen in the cross-section in panel B), a design in which the pressure drop is relatively higher along the left 1864 resulting in less flow of the fluid leaving from the left 1864 side than on the right 1866 side of the slot. Beneficially, the slot-die may be designed to vary the dispense flow rate with the radius of dispense location (on the substrate). Panel C illustrates a cross-sectional side view of a slot-die head 1863 dispensing fluid 1870. The slot-die head may comprise a cavity that branches out to multiple different output slot channels, which multiple channels can be designed to vary the dispense flow rate with the radius of dispense location on the substrate.

It will be appreciated that the dispense mechanisms and dispense tools for dispensing reagents and/or samples, as described herein, are not limited to those described in FIGS. 10A-10E.

One or more solutions or reagents may be delivered to a substrate by any of the delivery methods disclosed herein. Two or more solutions or reagents may be delivered to the substrate using the same or different delivery methods. Two or more solutions may be delivered to the substrate such that the time between contacting a solution or reagent and a subsequent solution or reagent is substantially similar for each region of the substrate contacted to the one or more solutions or reagents. A solution or reagent may be delivered as a single mixture. The solution or reagent may be dispensed in two or more component solutions. For example, each component of the two or more component solutions may be dispensed from a distinct nozzle. The distinct nozzles may dispense the two or more component solutions substantially simultaneously to substantially the same region of the substrate such that a homogenous solution forms on the substrate. Dispensing of each component of the two or more components may be temporally separated. Dispensing of each component may be performed using the same or different delivery methods. Direct delivery of a solution or reagent may be combined with spin-coating.

A solution may be incubated on the substrate for any desired duration (e.g., minutes, hours, etc.). In some embodiments, the solution may be incubated on the substrate under conditions that maintain a layer of fluid on the surface. One or more of the temperature of the chamber, the humidity of the chamber, the rotation of the substrate, and the composition of the fluid may be adjusted such that the layer of fluid is maintained during incubation. In some instances, during incubation, the substrate may be rotated at an rotational frequency of no more than 60 rpm, 50 rpm, 40 rpm, 30 rpm, 25 rpm, 20 rpm, 15 rpm, 14 rpm, 13 rpm, 12 rpm, 11 rpm, 10 rpm, 9 rpm, 8 rpm, 7 rpm, 6 rpm, 5 rpm, 4 rpm, 3 rpm, 2 rpm, 1 rpm or less. In some cases, the substrate may be rotating with a rotational frequency of about 5 rpm during incubation.

The substrate or a surface thereof may comprise other features that aid in solution or reagent retention on the substrate or thickness uniformity of the solution or reagent on the substrate. In some cases, the surface may comprise a raised edge (e.g., a rim) which may be used to retain solution on the surface. The surface may comprise a rim near the outer edge of the surface, thereby reducing the amount of the solution that flows over the outer edge.

The dispensed solution may comprise any sample or any analyte disclosed herein. The dispensed solution may comprise any reagent disclosed herein. In some cases, the solution may be a reaction mixture comprising a variety of components. In some cases, the solution may be a component of a final mixture (e.g., to be mixed after dispensing). In non-limiting examples, the solution can comprise samples, analytes, supports, beads, probes, nucleotides, oligonucleotides, labels (e.g., dyes), terminators (e.g., blocking groups), other components to aid, accelerate, or decelerate a reaction (e.g., enzymes, catalysts, buffers, saline solutions, chelating agents, reducing agents, other agents, etc.), washing solution, cleavage agents, combinations thereof, deionized water, and other reagents and buffers.

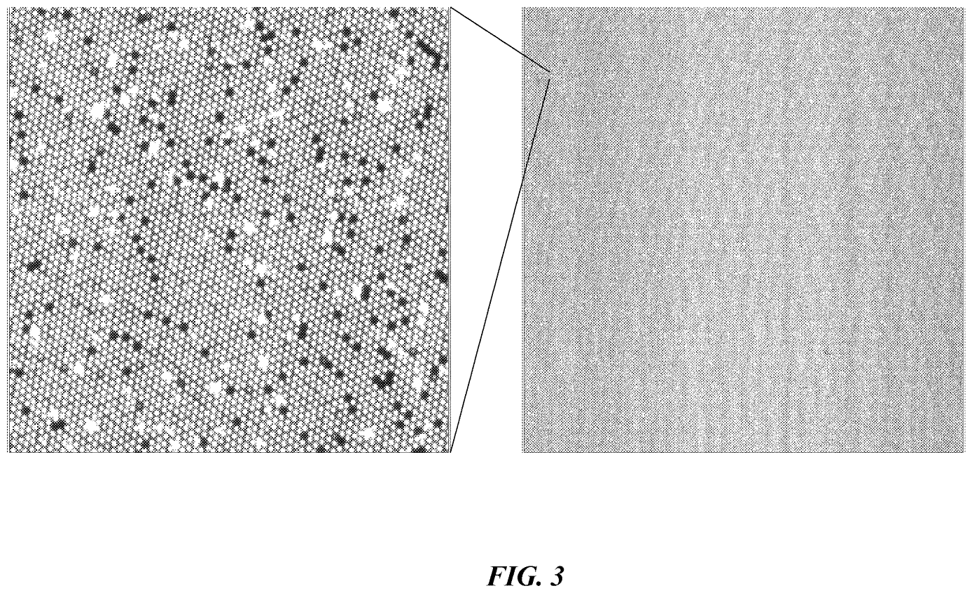

A sample may comprise beads, as described elsewhere herein, for example beads comprising nucleic acid colonies bound thereto. In some cases, an order of magnitude of at least and/or at most about 10¹, 10², 10³, 10⁴, 10⁵, 10⁶, 10⁷, 10⁸, 10⁹, 10¹⁰, 10¹¹, 10¹², 10¹³ or more beads may be loaded on the substrate, such as to immobilize to as many individually addressable locations. In some cases, the beads may be distinguishable from one another using a property of the beads, such as color, reflectance, anisotropy, brightness, fluorescence, etc. In some cases, as described elsewhere herein, different beads may comprise different tags (e.g., nucleic acid sequences) coupled thereto. For example, a bead may comprise an oligonucleotide molecule comprising a tag (e.g., barcode) that identifies a bead amongst a plurality of beads. FIG. 3 illustrates images of a portion of a substrate surface after loading a sample containing beads onto a substrate patterned with a substantially hexagonal lattice of individually addressable locations, where the right panel illustrates a zoomed-out image of a portion of a surface, and the left panel illustrates a zoomed-in image of a section of the portion of the surface. After sample loading, a “bead occupancy” may generally refer to the number of a type of individually addressable locations comprising at least one bead out of the total number of individually addressable locations of the same type. A bead “landing efficiency” may generally refer to the number of beads that bind to the surface out of the total number of beads dispensed on the surface.

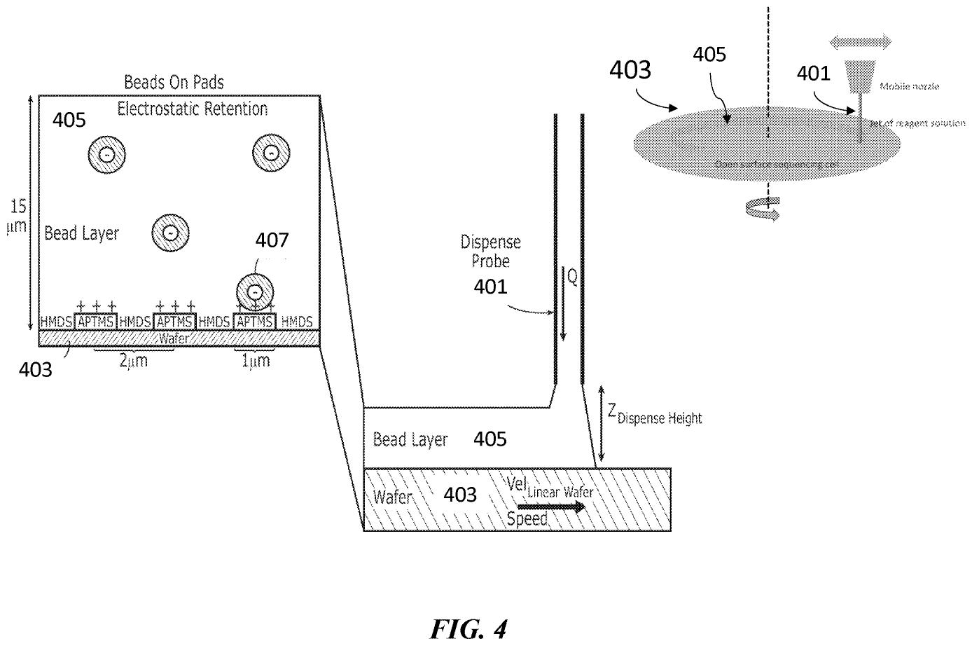

In some cases, beads may be dispensed to the substrate according to one or more systems and methods shown in FIG. 4. As shown in FIG. 4, a solution comprising beads may be dispensed from a dispense probe 401 (e.g., a nozzle) to a substrate 403 (e.g., a wafer) to form a layer 405. The dispense probe may be positioned at a height (“Z”) above the substrate. In the illustrated example, the beads are retained in the layer 405 by electrostatic retention, and may immobilize to the substrate at respective individually addressable locations. A set of beads in the solution may each comprise a population of amplified products (e.g., nucleic acid molecules) immobilized thereto, which amplified products accumulate to a negative charge on the bead. Otherwise, the beads may comprise reagents that have a negative charge. The substrate comprises alternating surface chemistry between distinguishable locations, in which a first location type comprises APTMS carrying a positive charge with affinity towards the negative charge of the amplified bead (e.g., a bead comprising amplified products immobilized thereto, and as distinguished from a negative bead which does not the comprise the same) or other bead comprising the negative charge, and a second location type comprises HMDS which has lower affinity and/or is repellant of the amplified bead or other bead comprising the negative charge. Within the layer 405 a bead may successfully land on a first location of the first location type (as in 407). In the illustrated example, the location size is 1 micron, the pitch between the different locations of the same location type (e.g., first location type) is 2 microns, and the layer has a depth of 15 micron. The top right panel illustrates that a reagent solution may be dispensed from the dispense probe 401 as the layer 405 along a path on an open surface of the substrate 403. The reagent may be dispensed on the surface in any desired pattern or path. The substrate 403 and the dispense probe 401 may move in any configuration with respect to each other to achieve any pattern (e.g., linear pattern, substantially spiral pattern, etc.).

Dispense mechanisms described herein may be operated by a fluid flow unit which may be controlled by one or more controllers, individually or collectively. The fluid flow unit may comprise any of the hardware and software components described with respect to the dispense mechanisms herein.

### Detection

An optical system comprising a detector may be configured to detect one or more signals from a detection area on the substrate prior to, during, or subsequent to, the dispensing of reagents to generate an output. Signals from multiple individually addressable locations may be detected during a single detection event. Signals from the same individually addressable location may be detected in multiple instances.

A signal may be an optical signal (e.g., fluorescent signal), electronic signal, or any detectable signal. The signal may be detected during rotation of the substrate or following termination of the rotation. The signal may be detected while the analyte is in fluid contact with a solution. The signal may be detected following washing of the solution. In some instances, after the detection, the signal may be muted, such as by cleaving a label from a probe and/or the analyte, and/or modifying the probe and/or the analyte. Such cleaving and/or modification may be performed by one or more stimuli, such as exposure to a chemical, an enzyme, light (e.g., ultraviolet light), or temperature change (e.g., heat). In some instances, the signal may otherwise become undetectable by deactivating or changing the mode (e.g., detection wavelength) of the one or more sensors, or terminating or reversing an excitation of the signal. In some instances, detection of a signal may comprise capturing an image or generating a digital output (e.g., between different images).

The operations of (i) directing a solution to the substrate and (ii) detection of one or more signals indicative of a reaction between a probe in the solution and an analyte immobilized to the substrate, may be repeated any number of times. Such operations may be repeated in an iterative manner. For example, the same analyte immobilized to a given location in the array may interact with multiple solutions in multiple cycles and for each iteration, the additional signals detected may provide incremental, or final, data about the analyte during the processing. For example, where the analyte is a nucleic acid molecule and the processing is sequencing, additional signals detected for each iteration may be indicative of one or more bases in the nucleic acid sequence of the nucleic acid molecule. In some cases, multiple solutions can be provided to the substrate without intervening detection events. In some cases, multiple detection events can be performed after a single flow of solution. In some instances, a washing solution, cleaving solution (e.g., comprising cleavage agent), and/or other solutions may be directed to the substrate between each operation, between each cycle, or a certain number of times for each cycle.

The optical system may be configured for continuous area scanning of a substrate during rotational motion of the substrate. The term “continuous area scanning (CAS),” as used herein, generally refers to a method in which an object in relative motion is imaged by repeatedly, electronically or computationally, advancing (clocking or triggering) an array sensor at a velocity that compensates for object motion in the detection plane (focal plane). CAS can produce images having a scan dimension larger than the field of the optical system. TDI scanning may be an example of CAS in which the clocking entails shifting photoelectric charge on an area sensor during signal integration. For a TDI sensor, at each clocking step, charge may be shifted by one row, with the last row being read out and digitized. Other modalities may accomplish similar function by high speed area imaging and co-addition of digital data to synthesize a continuous or stepwise continuous scan.

The optical system may comprise one or more sensors. The sensors may detect an image optically projected from the sample. The optical system may comprise one or more optical elements. An optical element may be, for example, a lens, tube lens, prism, mirror, wave plate, filter, attenuator, grating, diaphragm, beam splitter, diffuser, polarizer, depolarizer, retroreflector, spatial light modulator, or any other optical element. The system may comprise any number of sensors. In some cases, a sensor is any detector as described herein. In some examples, the sensor may comprise image sensors, CCD cameras, CMOS cameras, TDI cameras (e.g., TDI line-scan cameras), pseudo-TDI rapid frame rate sensors, or CMOS TDI or hybrid cameras. The optical system may further comprise any one or more optical sources (e.g., lasers, LED light sources, etc.). In some cases, where there are multiple sensors, the different sensors may image the same or different regions of the rotating substrate, in some cases simultaneously. Each sensor of the plurality of sensors may be clocked at a rate appropriate for the region of the rotating substrate imaged by the sensor, which may be based on the distance of the region from the center of the rotating substrate or the tangential velocity of the region. In some cases, multiple scan heads can be operated in parallel along different imaging paths (e.g., interleaved spiral scans, nested spiral scans, interleaved ring scans, nested ring scans). A scan head may comprise one or more of a detector element such as a camera (e.g., a TDI line-scan camera), an illumination source (e.g., as described herein), and one or more optical elements (e.g., as described herein).

The system may further comprise one or more controllers operatively coupled to the one or more sensors, individually or collectively programmed to process optical signals from the one or more sensors, such as for each region of the rotating substrate.

In some cases, the optical system may comprise an immersion objective lens. The immersion objective lens may be in contact with an immersion fluid that is in contact with the open substrate. The immersion fluid may comprise any suitable immersion medium for imaging (e.g., water, aqueous, organic solution). In some cases, an enclosure may partially or completely surround a sample-facing end of the optical imaging objective. The enclosure may be configured to contain the immersion fluid. The enclosure may not be in contact with the substrate; for example, a gap between the enclosure and the substrate may be filled by the fluid contained by the enclosure (e.g., the enclosure can retain the fluid via surface tension). In some cases, an electric field may be used to regulate a hydrophobicity of one or more surfaces of the container to retain at least a portion of the fluid contacting the immersion objective lens and the open substrate. In some cases, the immersion fluid may be continuously replenished or recycled via an inlet and outlet to the enclosure.

### High Throughput

An open substrate may be processed within a modular local sample processing environment. A barrier comprising a fluid barrier may be maintained between a sample processing environment and an exterior environment during certain processing operations, such as reagent dispensing and detecting. Systems and methods comprising a fluid barrier are described in further detail in U.S. Patent Pub. No. 20210354126A1, which is entirely incorporated herein by reference. A modular local sample processing environment may be defined by a chamber and a lid plate, where the lid plate is not in contact with the chamber, and the gap between the lid plate and the chamber may comprise the fluid barrier. The fluid barrier may comprise fluid (e.g., air) from the sample processing environment and/or the exterior environment and may have lower pressure than the sample processing environment, the external environment, or both. The fluid in the fluid barrier may be in coherent motion or bulk motion.

The sample processing environment may comprise therein a substrate, such as any substrate described elsewhere herein. Any operation performed on or with the substrate, as described elsewhere herein, may be performed within the sample processing environment while the fluid barrier is maintained. For example, the substrate may be rotated within the sample processing environment during various operations. In another example, fluid may be directed to the substrate while the substrate is in the sample processing environment, via a fluid handler (e.g., nozzle) that penetrates the lid plate into the sample processing environment. In another example, a detector can image the substrate while the substrate is in the sample processing environment, via a detector that penetrates the lid plate into the sample processing environment. Beneficially, the fluid barrier may help maintain temperature(s) and/or relative humidit(ies), or ranges thereof, within the sample processing environment during various processing operations.

The systems described herein, or any element thereof, may be environmentally controlled. For instance, the systems may be maintained at a specified temperature or humidity. For an operation, the systems (or any element thereof) may be maintained at a temperature of at least and/or at most 20 degrees Celsius (C), 25° C., 30° C., 35° C., 40° C., 45° C., 50° C., 55° C., 60° C., 65° C., 70° C., 75° C., 80° C., 85° C., 90° C., 95° C., 100° C., or more. Different elements of the system may be maintained at different temperatures or within different temperature ranges, such as the temperatures or temperature ranges described herein. Elements of the system may be set at temperatures above the dew point to prevent condensation. Elements of the system may be set at temperatures below the dew point to collect condensation.

While examples described herein provide relative rotational motion of the substrates and/or detector systems, the substrates and/or detector systems may alternatively or additionally undergo relative non-rotational motion, such as relative linear motion, relative non-linear motion (e.g., curved, arcuate, angled, etc.), and any other types of relative motion.

An open substrate may be retained in the same or approximately the same physical location during processing of an analyte and subsequent detection of a signal associated with the processed analyte. Alternatively, different operations on or with the open substrate may be performed in different stations disposed in different physical locations. For example, a first station may be disposed above, below, adjacent to, or across from a second station. In some cases, the different stations can be housed within an integrated housing. Alternatively, the different stations can be housed separately. In some cases, different stations may be separated by a barrier, such as a retractable barrier (e.g., sliding door). One or more different stations of a system, or portions thereof, may be subjected to different physical conditions, such as different temperatures, pressures, or atmospheric compositions. The open substrate may transition between different stations by transporting the sample processing environment comprising the chamber containing the open substrate between the different stations. One or more mechanical components or mechanisms, such as a robotic arm, elevator mechanism, actuators, rails, and the like, or other mechanisms may be used to transport the sample processing environment.

An environmental unit (e.g., humidifiers, heaters, heat exchangers, compressors, etc.) may be configured to regulate one or more operating conditions in each station. In some instances, each station may be regulated by independent environmental units. In some instances, a single environmental unit may regulate a plurality of stations. In some instances, a plurality of environmental units may, individually or collectively, regulate the different stations. An environmental unit may use active methods or passive methods to regulate the operating conditions. For example, the temperature may be controlled using heating or cooling elements. The humidity may be controlled using humidifiers or dehumidifiers. In some instances, a part of a particular station, such as within a sample processing environment, may be further controlled from other parts of the particular station. Different parts may have different local temperatures, pressures, and/or humidity. In one example, the delivery and/or dispersal of reagents may be performed in a first station having a first operating condition, and the detection process may be performed in a second station having a second operating condition different from the first operating condition. The first station may be at a first physical location in which the open substrate is accessible to a fluid handling unit during the delivery and/or dispersal processes, and the second station may be at a second physical location in which the open substrate is accessible to the detector system.

One or more modular sample environment systems (each having its own barrier system, e.g., fluid barrier) can be used between the different stations. In some instances, the systems described herein may be scaled up to include two or more of a same station type. For example, a sequencing system may include multiple processing and/or detection stations. FIGS. 5A-5B illustrate a system 300 that multiplexes two modular sample environment systems in a three-station system. In FIG. 5A, a first chemistry station (e.g., 320a) can operate (e.g., dispense reagents, e.g., to incorporate nucleotides to perform sequencing by synthesis) via at least a first operating unit (e.g., fluid dispenser 309a) on a first substrate (e.g., 311) in a first sample environment system (e.g., 305a) while substantially simultaneously, a detection station (e.g., 320b) can operate (e.g., scan) on a second substrate in a second sample environment system (e.g., 305b) via at least a second operating unit (e.g., detector 301), while substantially simultaneously, a second chemistry station (e.g., 320c) sits idle. An idle station may not operate on a substrate. An idle station (e.g., 320c) may be recharged, reloaded, replaced, cleaned, washed (e.g., to flush reagents), calibrated, reset, kept active (e.g., power on), and/or otherwise maintained during an idle time. After an operating cycle is complete, the sample environment systems may be re-stationed, as in FIG. 5B, where the second substrate in the second sample environment system (e.g., 305b) is re-stationed from the detection station (e.g., 320b) to the second chemistry station (e.g., 320c) for operation (e.g., dispensing of reagents, e.g., to incorporate nucleotides to perform sequencing by synthesis) by the second chemistry station, and the first substrate in the first sample environment system (e.g., 305a) is re-stationed from the first chemistry station (e.g., 320a) to the detection station (e.g., 320b) for operation (e.g., scanning) by the detection station. An operating cycle may be deemed complete when operation at each active, parallel station is complete. During re-stationing, the different sample environment systems may be physically moved (e.g., along the same track or dedicated tracks, e.g., rail(s) 307) to the different stations and/or the different stations may be physically moved to the different sample environment systems. One or more components of a station, such as modular plates 303a, 303b, 303c of plate 303 (e.g., lid plate) defining a particular station(s), may be physically moved to allow a sample environment system to exit the station, enter the station, or cross through the station. During processing of a substrate at station, the environment of a sample environment region (e.g., 315) of a sample environment system (e.g., 305a) may be controlled and/or regulated according to the station's requirements. After the next operating cycle is complete, the sample environment systems can be re-stationed again, such as back to the configuration of FIG. 5A, and this re-stationing can be repeated (e.g., between the configurations of FIGS. 5A and 5B) with each completion of an operating cycle until the required processing for a substrate is completed. In this illustrative re-stationing scheme, the detection station may be kept active (e.g., not have idle time not operating on a substrate) for all operating cycles by providing alternating different sample environment systems to the detection station for each consecutive operating cycle. Beneficially, use of the detection station is optimized. Based on different processing or equipment needs, an operator may opt to run the two chemistry stations substantially simultaneously while the detection station is kept idle.

Beneficially, different operations within the system may be multiplexed with high flexibility and control. For example, as described herein, one or more processing stations may be operated in parallel with one or more detection stations on different substrates in different modular sample environment systems to reduce or eliminate lag between different sequences of operations (e.g., chemistry first, then detection). The modular sample environment systems may be translated between the different stations accordingly to optimize efficient equipment use (e.g., such that the detection station is in operation almost 100% of the time). In some examples, at least 1, 2, 3, 4, 5, 6, 7, 8, 9, or more modules or stations of the sequencing system may be multiplexed. For example, 2 or more of the modules may each perform their intended function simultaneously or according to the methods described elsewhere herein. An example of this may comprise two-station multiplexing of an optics station and a chemistry station as described herein. Another example may comprise multiplexing three or more stations and process phases. For example, the method may comprise using staggered chemistry phases sharing a scanning station. The scanning station may be a high-speed scanning station. The modules or stations may be multiplexed using various sequences and configurations.

The nucleic acid sequencing systems and optical systems described herein (or any elements thereof) may be combined in a variety of architectures.

Sample processing systems, their various components, and methods are additionally described in U.S. Pat. Pub. Nos. 2020/0326327A1, 2021/0277464A1, 2023/0279487A1, and International Pat. Pub. No. WO2023/192403, each of which is entirely incorporated by reference herein for all purposes.

### Substrate Support Configurations

An open substrate may be supported in the system via the bottom surface of the substrate. For example, as illustrated in FIGS. 5A-5B, a substrate may be supported by a chuck (e.g., vacuum chuck) affixed to the bottom surface of the substrate. Analytes (e.g., samples) may be deposited and processed on the top surface of the substrate as the chuck supports the substrate from the bottom surface. The substrate may be rotated via the chuck from the bottom surface. This configuration may be referred to herein as an ‘upright configuration.’ Beneficially, in the upright configuration, the entire surface area or almost the entire surface area of the top surface of the substrate may be an active surface and be available for sample loading and processing. Further, the substrate may rest against the chuck via its weight working with gravitational force. As used herein, unless explicitly defined or described otherwise, the term “bottom” is generally used to describe an object that is positioned below or lower (towards gravitational force) than another object (e.g., than a top object), and the term “top” is generally used to describe an object that is positioned above or higher (opposite gravitational force) than another object (e.g., than a bottom object).

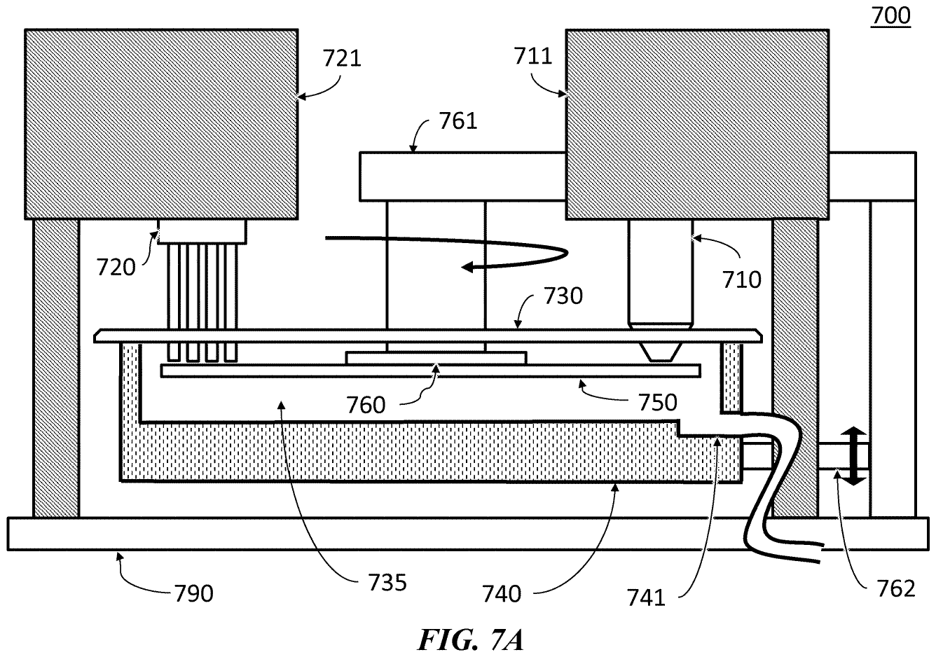

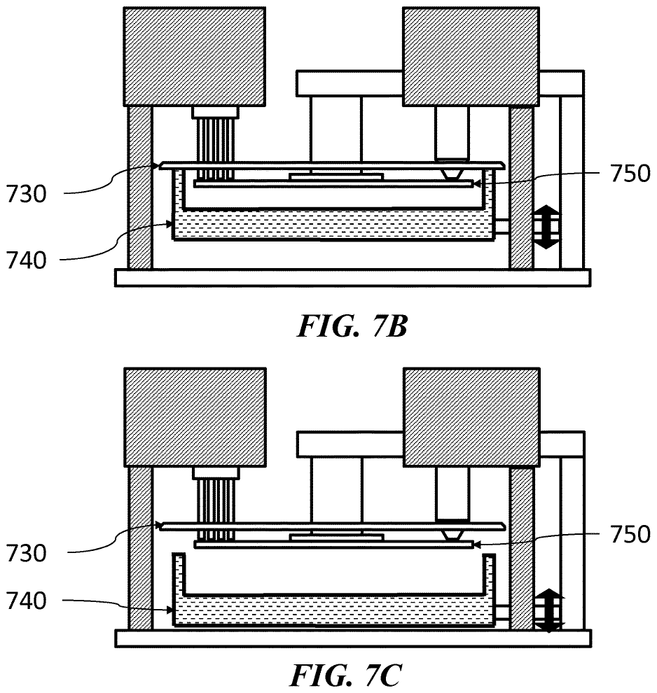

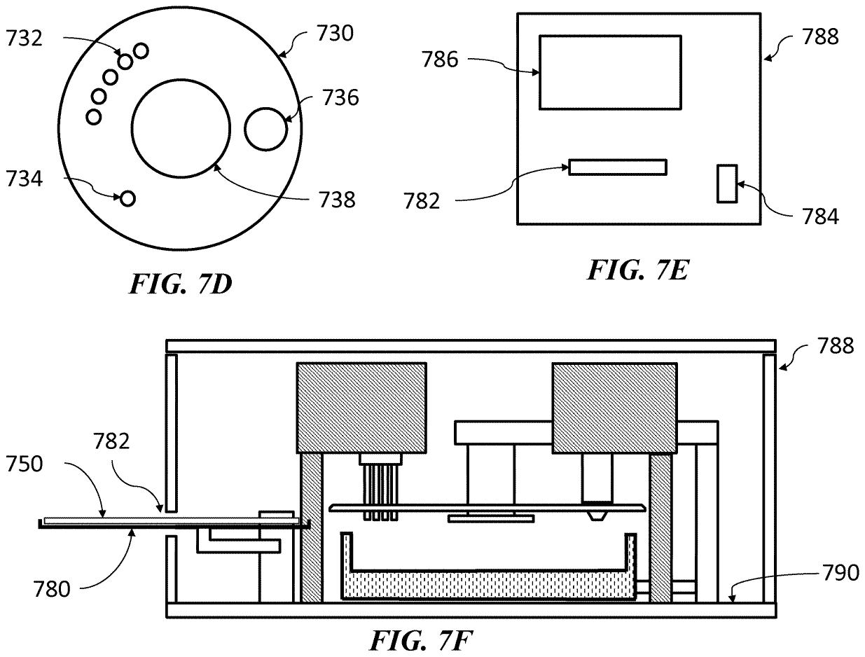

Alternatively, an open substrate may be supported in the system via the top surface of the substrate. For example, as illustrated in FIGS. 7A-7C, a substrate may be supported by a chuck (e.g., vacuum chuck) affixed to the top surface of the substrate via a cantilever-like structure. Analytes (e.g., samples) may be deposited and processed on a remaining surface area of the top surface (reduced active surface) of the substrate as the chuck supports the substrate from the top surface. The substrate may be rotated via the chuck from the top surface. This configuration may be referred to herein as a ‘cantilever configuration.’ Beneficially, in the cantilever configuration, the space below the substrate may be freed up and hardware or electronics (e.g., vacuum chuck) that are otherwise susceptible to contamination or damage from dripping fluids, drainage, overflow, leakage, or other wet contact from the sample processing operations can be moved elsewhere. Furthermore, the rigidity or stiffness of the substrate support and other components in the system may be increased by allowing flexible positioning of the cantilever base relative to neighboring hardware bases (e.g., optics base) such that vibrations and other kinetic disruptions from the neighboring hardware are dampened or sufficiently isolated.

FIG. 7A illustrates example cantilever configurations of an open substrate system. A system 700 may comprise a system base 790 to which is affixed a fluidics station 721, an optics station 711, and a cantilever structure 761. The system 700 may be a sequencing system. The fluidics station 721 may comprise one or more first operation units 720 (e.g., fluid dispensers). The optics station 711 may comprise one or more second operation units 710 (e.g., detectors). The cantilever structure 761 may comprise a cantilever arm with a chuck 760 disposed at the end of the arm. The chuck 760 may be configured to affix to a top surface of a substrate 750 and support the substrate 750. The substrate may be configured to be housed in a sample environment system comprising a sample environment 735 defined by a chamber 740 and lid plate 730. The sample environment system may comprise a fluid barrier as described elsewhere herein during operation (e.g., maintain a physical gap between the chamber and lid plate, which gap comprises fluid). The sample environment system may not comprise a fluid barrier, such that during operation the lid plate 730 contacts and closes the chamber 740 without a gap. The sample processing environment 735 may comprise therein the substrate 750, and any operation performed on or with the substrate, as described elsewhere herein, may be performed within the sample processing environment. For example, the substrate 750 may be rotated, e.g., via the chuck 760, in the sample processing environment 735 during various operations. The chamber 740 may comprise a drainage system 741, for example comprising an active and/or passive drain, configured to direct fluids out of the sample environment system.

The chuck 760, one or more first operation units 720, and one or more second operation units 710 may penetrate the lid plate 730 into the sample environment 735 to perform various operations on or with the substrate 750. FIG. 7D illustrates a schematic top view of lid plate 730. The lid plate 730 comprises first openings 732 to allow for penetration of the one or more first operation units 720, second opening 736 to allow for penetration of the one or more second operation units 710, third opening 738 for penetration of the chuck 760, and fourth opening 734 for penetration of an additional operation unit (not depicted in FIG. 7A) such as a sample dispenser. The openings in the lid plate 730 that permit such penetrations may be sealed (e.g., hermetically sealed) via the penetrating objects and/or other objects such as gaskets, sealants, or lids to seal the sample environment 735 from an exterior or ambient environment. In some cases, after a particular operation, an opening in the lid plate may not be in use and the opening may be sealed before and after an operation unit uses the opening.

The chamber 740 on its inner surface(s) may comprise one or more fluid nozzles (not illustrated) fluidically coupled to a hot fluid supply and cold fluid supply. In some cases, a fluid nozzle(s) of the one or more fluid nozzles may be coupled to only one of a hot fluid supply or a cold fluid supply. In some cases, a fluid nozzle(s) of the one or more fluid nozzles may be coupled to both the hot fluid supply and the cold fluid supply, e.g., via valve(s). The one or more fluid nozzles may be configured to direct fluid to a bottom surface of the substrate 750 during one or more processing operations to thermocycle the substrate 750. For example, hot fluid may be directed at the substrate to increase and/or maintain the temperature or temperature range of the substrate and cold fluid may be directed at the substrate to decrease and/or maintain the temperature or temperature range of the substrate. In another example, a combination of the hot fluid and the cold fluid (which may be mixed prior to dispensing to the substrate, and/or dispensed separately) may be directed at the substrate to increase, decrease, and/or maintain the temperature or temperature range of the substrate. The cold fluid supply may comprise fluid maintained at a temperature or temperature range below at most about 15, 16, 17, 18, 19, 20, 21, 22, 23, 24, 25, 30, 35, 40, 45, 50, 55, 60, 65, 70 degrees Celsius (° C.) or less. The hot fluid supply may comprise fluid maintained at a temperature or temperature range below at most about 30, 35, 40, 45, 50, 55, 60, 65, 70, 75, 80, 85, 90, 95 degrees Celsius (° C.) or more. For example, a surface temperature of the top surface of the substrate 750 may be adjusted to and/or maintained at temperatures or temperature ranges comprising about, at least about, and/or at most about 15, 16, 17, 18, 19, 20, 21, 22, 23, 24, 25, 30, 35, 40, 45, 50, 55, 60, 65, 70, 75, 80, 85, 90, 95 degrees Celsius (C) or more. In some cases, via thermocycling, the surface temperature may be increased by at least 20° C. in less than about 60, 55, 50, 45, 40, 35, 30 or less seconds. In some cases, via thermocycling, the temperature may be increased by at least 30° C. in less than about 60, 55, 50, 45, 40, 35, 30 or less seconds. In some cases, via thermocycling, the temperature may be increased by at least 40° C. in less than about 60, 55, 50, 45, 40, 35, 30 or less seconds. In some cases, via thermocycling, the surface temperature may be decreased by at least 20° C. in less than about 60, 55, 50, 45, 40, 35, 30 or less seconds. In some cases, via thermocycling, the temperature may be decreased by at least 30° C. in less than about 60, 55, 50, 45, 40, 35, 30 or less seconds. In some cases, via thermocycling, the temperature may be decreased by at least 40° C. in less than about 60, 55, 50, 45, 40, 35, 30 or less seconds. The surface temperature may refer to an average surface temperature measured across the substrate surface. Alternatively, the surface temperature may refer to the temperature of a fixed measurement point on the substrate surface. The inner surface(s) of the chamber 740, such as the bottom surface and/or walls, may comprise any number of such fluid nozzles, such as about, at least about, and/or at most about 1, 2, 3, 4, 5, 6, 7, 8, 9, 10, 15, 20, 25, 30 or more fluid nozzles.

The chamber 740 on its inner surface(s) may comprise one or more additional fluid nozzles (not illustrated) fluidically coupled to a cleaning solution. The one or more additional fluid nozzles may be configured to direct cleaning solution to the ceiling of the sample environment 735 (or bottom surface of the lid plate 730) to clean the ceiling. Alternatively or in addition, the one or more fluid nozzles used for thermocycling may also be fluidically coupled to a cleaning solution and be configured to direct cleaning solution to the ceiling.

FIGS. 7B and 7C illustrate the sample environment system in the cantilever configuration in the closed and open position, respectively. The chamber 740 may be actuatable along a z-axis (up and down) via mechanical arm 762 to assemble with or disassemble from the lid plate 730, to allow for loading and/or unloading of the substrate 750 to the chuck 760 in the sample environment 735. Alternatively or in addition, the lid plate 730 may be actuatable (e.g., via mechanical arm) to assemble with or disassemble with the chamber 740. Upon separation of the chamber 740 and the lid plate 730, in the open position, the substrate 750 may enter or exit the sample environment 735 via a gap between the chamber and the lid plate. After entry or exit of the substrate 750, the chamber 740 and the lid plate 730 may be actuated back to the closed position.

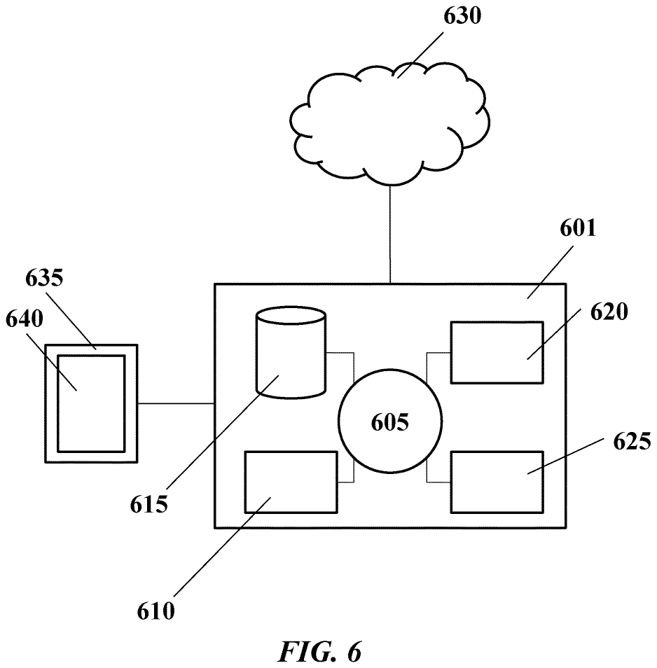

FIG. 7E and FIG. 7F illustrate a schematic front view and cross-sectional side view of a packaged system, e.g., of a sequencing system. The system 700 may be packaged within exterior walls 788. One or more walls of the exterior walls may comprise a user interface 786, a substrate slot 782, and a reagent slot 784. The system 700 may comprise a substrate handler 780, comprising a substrate tray, which receives the substrate 750 via the substrate slot 782 and loads the substrate 750 to the chuck 760 in the sample environment 735 when the sample environment system is in the open position. In reverse, the substrate handler 780 may unload the substrate 750 from the chuck 760 and deliver the substrate 750 outside the exterior walls 788 (e.g., to the user) via the substrate slot 782. The reagent slot 784 may receive one or more reagents into the system 700 such as via tube, cartridge, tray, rack, or other form. Once inserted, one or more operation units in the system 700, such as those of a sample loading station and/or chemistry station, may have access to the reagents. It will be appreciated that the packaged system may comprise additional slots, e.g., additional reagent slots. The user interface 786 may comprise a display, speakers, and/or other user input/output devices. The system 700 may comprise a computer system, such as one described with respect to FIG. 6, which is in communication with the user interface 786.

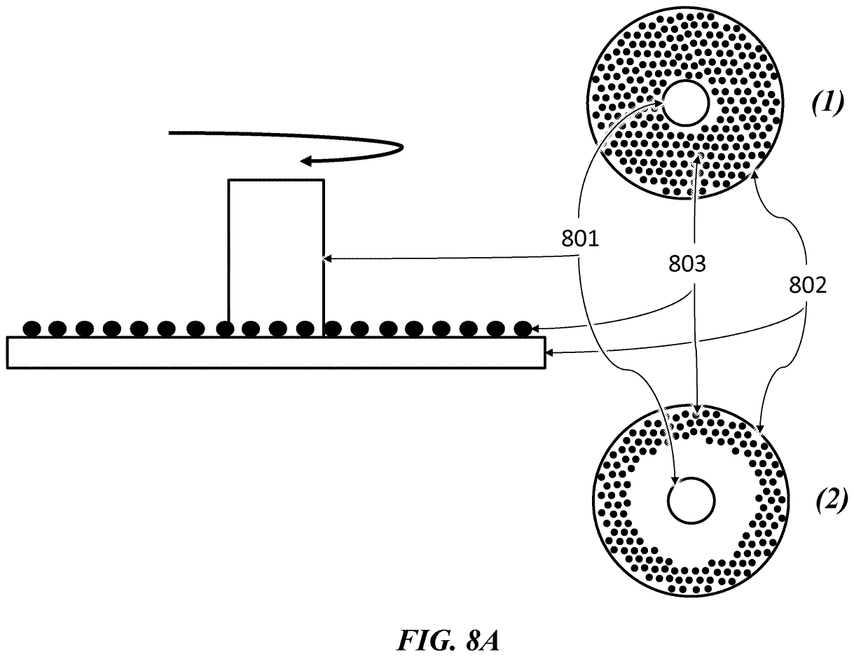

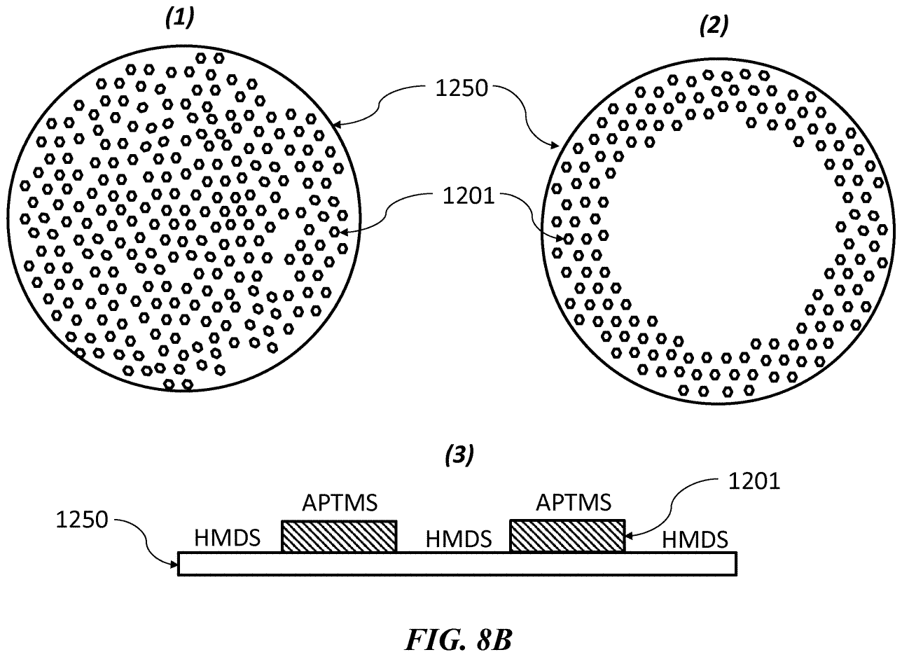

In the cantilever configuration, the active surface of the open substrate is reduced by the area the chuck is affixed to on the top surface of the open substrate. In some cases, all or almost all of the remaining surface area may be used as an active surface. In some cases, a smaller periphery of the remaining surface area may be used as an active surface. FIG. 8A shows different active surface areas on an open substrate. Where a chuck 801 is affixed to a top surface of the substrate 802, the sample 803 may be deposited to different active surface areas. As shown in panel (1), all or almost all of the remaining area of a top surface of the substrate 802, subtracting the chuck 801, may be used as an active surface and the sample 803 is loaded onto all radii from an outer edge of the chuck to an outer edge of the substrate. As shown in panel (2), a smaller radii range of the substrate 802 may be used as an active surface, and the sample 803 is loaded onto all radii from a radius that is some distance outwards from the outer edge of the chuck to the outer edge of the substrate. It will be appreciated that the radii range may start or end at a radius that is not an outer edge of the substrate or outer edge of the chuck.

In some cases, the active surface may be defined by the surface area between the outer edge of the substrate and a radius of about, at least about, and/or at most about 0.1, 0.2, 0.3, 0.4, 0.5, 0.6, 0.7, 0.8, 0.9, 1.0, 2.0, 3.0, 4.0, 5.0, 6.0, 7.0, 8.0, 9.0, 10.0 centimeters(s) or more inwards from the outer edge of the substrate. Alternatively or in addition, the active surface may be defined by the surface area between the outer edge of the chuck and a radius of about, at least about, and/or at most about 0.1, 0.2, 0.3, 0.4, 0.5, 0.6, 0.7, 0.8, 0.9, 1.0, 2.0, 3.0, 4.0, 5.0, 6.0, 7.0, 8.0, 9.0, 10.0 centimeters(s) or more outwards from the outer edge of the chuck. Alternatively or in addition, the active surface may be defined by the surface area between the outer edge of the substrate and a radius of about, at least about, and/or at most about 0.1, 0.2, 0.3, 0.4, 0.5, 0.6, 0.7, 0.8, 0.9, 1.0, 2.0, 3.0, 4.0, 5.0, 6.0, 7.0, 8.0, 9.0, 10.0 centimeters(s) or more outwards from the outer edge of the chuck. Alternatively or in addition, the active surface may be defined by the surface area between a radius of about, at least about, and/or at most about 0.1, 0.2, 0.3, 0.4, 0.5, 0.6, 0.7, 0.8, 0.9, 1.0, 2.0, 3.0, 4.0, 5.0, 6.0, 7.0, 8.0, 9.0, 10.0 centimeters(s) or more inwards from the outer edge of the substrate and a radius of about, at least about, and/or at most about 0.1, 0.2, 0.3, 0.4, 0.5, 0.6, 0.7, 0.8, 0.9, 1.0, 2.0, 3.0, 4.0, 5.0, 6.0, 7.0, 8.0, 9.0, 10.0 centimeters (s) or more outwards from the outer edge of the chuck. Alternatively or in addition, the active surface may be defined by the surface area between a radii difference of about, at least about, and/or at most about 0.1, 0.2, 0.3, 0.4, 0.5, 0.6, 0.7, 0.8, 0.9, 1.0, 2.0, 3.0, 4.0, 5.0, 6.0, 7.0, 8.0, 9.0, 10.0 centimeters(s).

In some cases, an active surface may be defined by surface chemistry such that the surface chemistry between active surfaces and non-active surfaces are different. In some cases, an active surface may be further distinguished within the active surface with surface chemistry. For example, where 50% of a substrate surface is an active surface, 80% of the 50% active surface area (or 40% of the total substrate surface) may be further distinguished with surface chemistry. FIG. 8B illustrates different active areas that have distinct surface chemistry. It will be appreciated that a type of surface chemistry can comprise any pattern, any array, and/or random arrangement, of any number of moieties. For example, a type of surface chemistry may comprise a single type of moiety or element. In another example, a type of surface chemistry may comprise two types of moieties: e.g., type A and type B. The two types of moieties may be alternating, one type may be spotted onto another type, or one type may be spotted and another type filling in the remaining area, etc. In another example, a type of surface chemistry may comprise three or more types of moieties in any arrangement. As described elsewhere herein, the substrate surface may be patterned or unpatterned and/or texture or untextured. Panels (1)-(3) illustrate an example surface chemistry 1201 that comprise two moieties: type A (e.g., APTMS) and type B (e.g., HMDS), in which type A moieties are spotted in distinct locations, and remaining locations comprise type B moieties. In some cases, the surface chemistry and/or a moiety of a surface chemistry may define individually addressable locations as described elsewhere herein. In some cases, the surface chemistry and/or a moiety of a surface chemistry may function as one or more, or a part of a, binder(s) that interact with samples and/or reagents. Panel (1) illustrates a surface of a substrate 1250 that is uniformly covered with surface chemistry 1201, where type A moieties are randomly located to define different individually addressable locations. Panel (2) illustrates a surface of a substrate 1250 that is only partially covered with surface chemistry 1201, at the periphery of the substrate 1250. For example, the surface chemistry 1201 portion of the surface may define an active area of the substrate. In another example, the substrate may have a larger active area, and the surface chemistry 1201 portion of the surface may define a sub-area within an active area of the substrate.

Alternatively or in addition, a non-active surface may be defined by surface chemistry such that the surface chemistry between active surfaces and non-active surfaces are different. For example, in the example of panel (2) of FIG. 8B, the surface chemistry 1201 portion of the surface may define a non-active area of the substrate, and the active area of the substrate may be the inner radii of the substrate (depicted as blank area in the figure).

FIGS. 5A-5B illustrate open substrate systems in which different operating stations are positioned at different locations and a modular sample environment travels between the different locations of the different operating stations. That is, the modular sample environment comprising the substrate has to be positioned at a first station to be operated on by a first operating unit (e.g., fluid dispenser) of the first station, and then move to a second location to be positioned at a second station to be operated on by a second operating unit (e.g., detector) of the second station. Alternatively, an open substrate system may comprise different operating stations that are accessible by a sample environment such that at a timepoint, a single substrate is accessible by multiple operating units form multiple stations. For example, different stations may be positioned to operate at different radial portions of a substrate relative to a rotating axis or central axis. FIGS. 7A-7C and FIG. 7F illustrate open substrate systems in which two operating stations, e.g., a fluidics station and an optics station, are positioned to operate different radial portions of the substrate, at opposite ends of the substrate. Beneficially, positioning different stations at different radial portions of a substrate within the same sample environment eliminates the need to move the sample environment (and the substrate therein) between different station locations-the substrate need only rotate about a rotating axis for a radial portion of the substrate to access different stations. The sample environment may be stationary. For clarity, the term “radial” is with respect to a rotating axis, and a substrate may be circular or non-circular and the rotating axis may be the central axis or any other non-central axis.

Any number of operating stations and/or operating units may be positioned to operate on a single sample environment or single substrate. For example, about, at least about, and/or at most about 1, 2, 3, 4, 5, 6, 7, 8 or more stations may be positioned to operate on a single sample environment or single substrate. The stations may be the same type of stations or different types of stations. Example stations include chemistry stations, fluidics stations, washing stations, sample loading stations, detection stations, and the like. In such configurations, different operating units (e.g., fluidic dispensers, detectors) may operate simultaneously or at different time points. In an example, a first station is active (e.g., operates) while a second station is inactive (e.g., not operating) for a predetermined first time period or for a predetermined number of rotations of the substrate, after which the second station is active while the first station is inactive for a predetermined second time period or for a predetermined number of rotations of the substrate. In some cases, only one station and/or operating unit may be active at a time such that all other stations and/or operating units are inactive during another station and/or operating unit's operations. In some cases, two or more stations and/or operating units may be active at the same time such that some stations are active while some stations are inactive. In some cases, all stations may be active simultaneously. Operation of a station or operating unit may be programmed according to any useful functions or variables, such as to operate for a predetermined time period, to operate for a predetermined number of rotations of the substrate, to operate until completion of an active (e.g., dispensing a certain volume of a reagent), to operate upon completion of operation of another station or operation unit, to operate upon or until completion of any other activity or condition(e.g., data analysis, data transfer, user input, user output, etc.), etc.

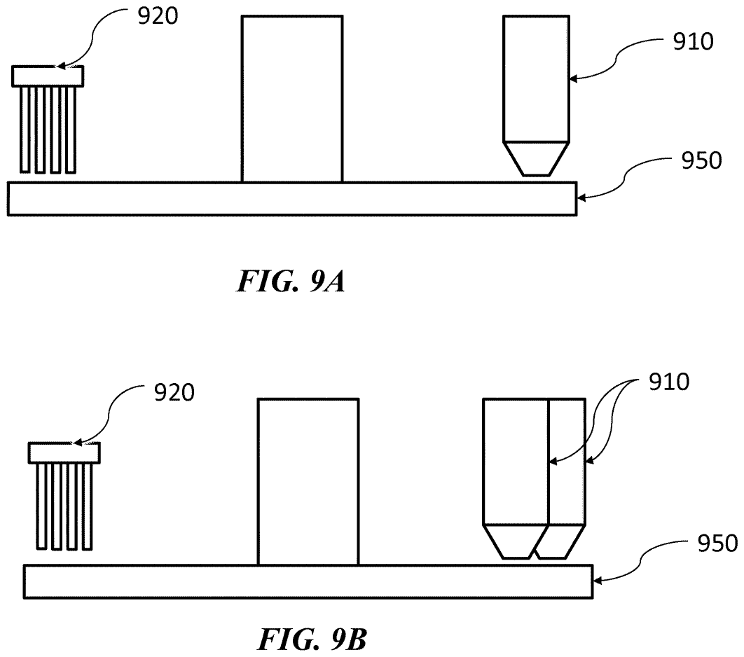

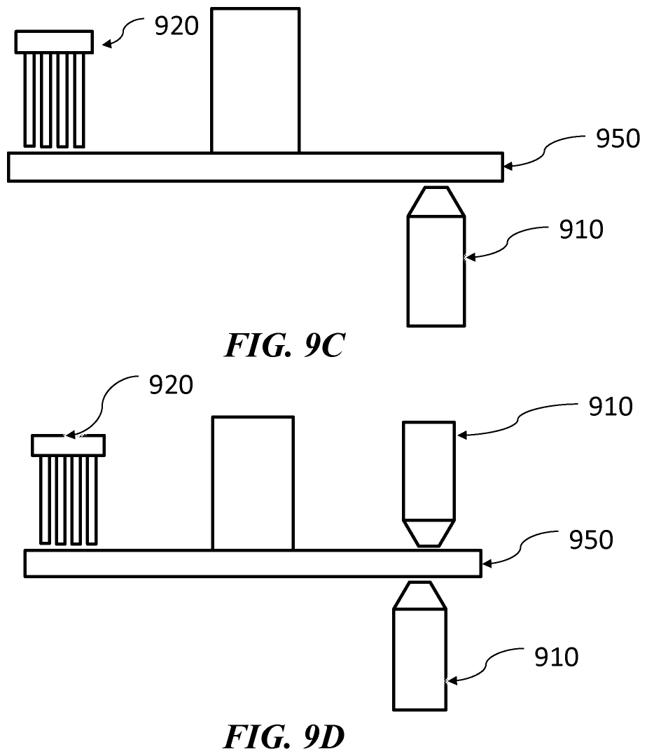

FIGS. 9A-9D show different placement schemes of chemistry and detection stations in the cantilever configuration. As shown in FIG. 9A, a chemistry station 920 and a detection station 910 may be placed on substantially opposing radial portions of a substrate 950. As shown in FIG. 9B, a chemistry station 920 and multiple detection stations (e.g., 910) or a single detection station with multiple objectives may be placed on substantially opposing radial portions of a substrate 950. Where there is a single objective, the single objective may be stationary and positioned at a fixed radius from the rotational axis. The single objective may be movable with respect to the substrate and/or the substrate may be movable with respect to the objective across varying radii from the rotational axis, such as to image and/or scan substrate areas between wider radii. Where there are multiple objectives, the objectives may be positioned at the same radius or at different radii. One or more, or all of the multiple objectives may be stationary and/or be movable across varying radii for efficient imaging and/or scanning. Different objective and imaging/scanning configurations that are available with a rotating substrate are described elsewhere herein. As shown in FIG. 9C, a chemistry station 920 and a detection station 910 may be placed on substantially opposing radial portions of a substrate 950, where the objective of the detection station is positioned below the bottom surface of the substrate. In some cases, the substrate may be transparent or have some degree of transparency or translucency to permit a detector to collect signals derived from analytes placed on the top surface of the substrate. For example, the substrate may be a transparent glass material. As shown in FIG. 9D, a chemistry station 920 and a detection station 910 may be placed on substantially opposing radial portions of a substrate 950, where a first objective of the detection station is positioned above the top surface of the substrate and a second objective of the detection station is positioned below the bottom surface of the substrate. It will be appreciated that the cantilever configuration also allows a modular sample environment to travel between single stations that are positioned at different locations, as depicted in FIGS. 5A-5B.

Sample processing systems of the present disclosure, such as of upright configuration or cantilever configuration, may be packaged into a container of any form factor (shape or size). In some cases, the systems may be designed for laboratory use and be contained within a freestanding cabinet or benchtop form factor. In some cases, a maximum dimension of the container of a system may be about or at most about 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5 meters (m) or less. In some cases, a maximum dimension of the container of a system may be about or at most about 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5 meters (m) or less. In some cases, for a benchtop form factor, a maximum height of the container of a system may be about or at most about 0.5, 0.6, 0.7, 0.8, 0.9, 1.0, 1.1, 1.2, 1.3, 1.4, 1.5 meters (m) or less. In some cases, for a benchtop form factor, a maximum depth of the container of a system (depth is along depth of a lab bench for example) may be about or at most about 0.5, 0.6, 0.7, 0.8, 0.9, 1.0, 1.1, 1.2 meters (m) or less. In some cases, for a benchtop form factor, a maximum volume of the container of a system may be about or at most about 1.0, 1.1, 1.2, 1.3, 1.4, 1.5 , 1.6, 1.7, 1.8, 1.9, 2.0, 2.1, 2.2, 2.3, 2.4, 2.5 cubic meters (m³) or less.

### Computer Systems

The present disclosure provides computer control systems that are programmed to implement methods of the disclosure. FIG. 6 shows a computer system 601 that is programmed or otherwise configured to implement methods of the disclosure, such as to control the systems described herein (e.g., reagent dispensing, detecting, etc.) and collect, receive, and/or analyze sequencing information. The computer system 601 can be an electronic device of a user or a computer system that is remotely located with respect to the electronic device. The electronic device can be a mobile electronic device.

The computer system 601 includes a central processing unit (CPU, also “processor” and “computer processor” herein) 605, which can be a single core or multi core processor, or a plurality of processors for parallel processing. The computer system 601 also includes memory or memory location 610 (e.g., random-access memory, read-only memory, flash memory), electronic storage unit 615 (e.g., hard disk), communication interface 620 (e.g., network adapter) for communicating with one or more other systems, and peripheral devices 625, such as cache, other memory, data storage and/or electronic display adapters. The memory 610, storage unit 615, interface 620 and peripheral devices 625 are in communication with the CPU 605 through a communication bus (solid lines), such as a motherboard. The storage unit 615 can be a data storage unit (or data repository) for storing data. The computer system 601 can be operatively coupled to a computer network (“network”) 630 with the aid of the communication interface 620. The network 630 can be the Internet, an isolated or substantially isolated internet and/or extranet, or an intranet and/or extranet that is in communication with the Internet. The network 630 in some cases is a telecommunication and/or data network. The network 630 can include one or more computer servers, which can enable distributed computing, such as cloud computing. The network 630, in some cases with the aid of the computer system 601, can implement a peer-to-peer network, which may enable devices coupled to the computer system 601 to behave as a client or a server. The CPU 605 can execute a sequence of machine-readable instructions, which can be embodied in a program or software. The instructions may be stored in a memory location, such as the memory 610. The instructions can be directed to the CPU 605, which can subsequently program or otherwise configure the CPU 605 to implement methods of the present disclosure. Examples of operations performed by the CPU 605 can include fetch, decode, execute, and writeback. The CPU 605 can be part of a circuit, such as an integrated circuit. One or more other components of the system 601 can be included in the circuit. In some cases, the circuit is an application specific integrated circuit (ASIC).

The storage unit 615 can store files, such as drivers, libraries and saved programs. The storage unit 615 can store user data, e.g., user preferences and user programs. The computer system 601 in some cases can include one or more additional data storage units that are external to the computer system 601, such as located on a remote server that is in communication with the computer system 601 through an intranet or the Internet.

The computer system 601 can communicate with one or more remote computer systems through the network 630. For instance, the computer system 601 can communicate with a remote computer system of a user. Examples of remote computer systems include personal computers (e.g., portable PC), slate or tablet PC's (e.g., Apple® iPad, Samsung® Galaxy Tab), telephones, Smart phones (e.g., Apple® iPhone, Android-enabled device, Blackberry®), or personal digital assistants. The user can access the computer system 601 via the network 630.

Methods as described herein can be implemented by way of machine (e.g., computer processor) executable code stored on an electronic storage location of the computer system 601, such as, for example, on the memory 610 or electronic storage unit 615. The machine executable or machine readable code can be provided in the form of software. During use, the code can be executed by the processor 605. In some cases, the code can be retrieved from the storage unit 615 and stored on the memory 610 for ready access by the processor 605. In some situations, the electronic storage unit 615 can be precluded, and machine-executable instructions are stored on memory 610. The code can be pre-compiled and configured for use with a machine having a processor adapted to execute the code or can be compiled during runtime. The code can be supplied in a programming language that can be selected to enable the code to execute in a pre-compiled or as-compiled fashion.

Aspects of the systems and methods provided herein, such as the computer system 601, can be embodied in programming. Various aspects of the technology may be thought of as “products” or “articles of manufacture” typically in the form of machine (or processor) executable code and/or associated data that is carried on or embodied in a type of machine readable medium. Machine-executable code can be stored on an electronic storage unit, such as memory (e.g., read-only memory, random-access memory, flash memory) or a hard disk. “Storage” type media can include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical, and electromagnetic waves, such as used across physical interfaces between local devices, through wired and optical landline networks and over various air-links. The physical elements that carry such waves, such as wired or wireless links, optical links or the like, also may be considered as media bearing the software. As used herein, unless restricted to non-transitory, tangible “storage” media, terms such as computer or machine “readable medium” refer to any medium that participates in providing instructions to a processor for execution.

Hence, a machine readable medium, such as computer-executable code, may take many forms, including but not limited to, a tangible storage medium, a carrier wave medium or physical transmission medium. Non-volatile storage media include, for example, optical or magnetic disks, such as any of the storage devices in any computer(s) or the like, such as may be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

The computer system 601 can include or be in communication with an electronic display 635 that comprises a user interface (UI) 640 for providing, for example, control options of a sequencing system or displaying results of a nucleic acid sequence (e.g., sequence reads). Examples of UI's include, without limitation, a graphical user interface (GUI) and web-based user interface.

Methods and systems of the present disclosure can be implemented by way of one or more algorithms. An algorithm can be implemented by way of software upon execution by the central processing unit 605.

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations or equivalents. It is intended that the following claims define the scope of the invention and that methods and structures within the scope of these claims and their equivalents be covered thereby.

1.-37. (canceled)

38. A system for processing analytes, comprising:
a chamber;
a lid plate, wherein the lid plate and chamber are configured to close and open with respect to each other, wherein the chamber and lid plate enclose a sample environment configured to receive a substrate therein;
a detector that penetrates the lid plate into the sample environment;
a fluid dispenser that penetrates the lid plate into the sample environment; and
a chuck that penetrates the lid plate into the sample environment, wherein the chuck is configured to affix to a top surface of the substrate to support the substrate in the sample environment.

39. The system of claim 38, wherein the chuck is configured to rotate the substrate within the sample environment.

40. The system of claim 38, wherein the chuck is a vacuum chuck.

41. The system of claim 38, wherein an inner surface of the chamber comprises one or more nozzles fluidically coupled to a hot fluid supply and a cold fluid supply, wherein hot fluid in the hot fluid supply is maintained at temperatures or temperature ranges that are higher than those of cold fluid in the cold fluid supply, and wherein the one or more nozzles are configured to direct cold fluid, hot fluid, or both onto a bottom surface of the substrate.

42. The system of claim 38, wherein an inner surface of the chamber comprises one or more additional nozzles fluidically coupled to a cleaning solution, and wherein the one or more additional nozzles are configured to direct the cleaning solution to a bottom surface of the lid plate.

43. The system of claim 38, further comprising a mechanical arm coupled to the chamber, wherein the mechanical arm is configured to move the chamber relative to the lid plate to adjust the chamber between an open configuration and closed configuration.

44. The system of claim 38, further comprising a container comprising exterior walls enclosing the chamber, the lid plate, the detector, the fluid dispenser, and the chuck.

45. The system of claim 44, wherein one or more walls of the container comprises a substrate slot configured to permit the substrate to enter or exit the container.

46. The system of claim 44, wherein one or more walls of the container comprises a reagent slot configured to permit a reagent cartridge to enter or exit the container.

47. The system of claim 38, wherein the fluid dispenser is configured to dispense fluid to the top surface of the substrate.

48. The system of claim 38, wherein the detector is configured to collect one or more signals from the top surface of the substrate.

49. The system of claim 38, further comprising the substrate, wherein the substrate comprises on the top surface a plurality of analytes immobilized thereon.

50. The system of claim 49, wherein the plurality of analytes comprises a plurality of nucleic acid molecules.

51. The system of claim 50, wherein the plurality of nucleic acid molecules is directly immobilized to the top surface.

52. The system of claim 50, wherein the plurality of nucleic acid molecules is coupled to a plurality of beads, and wherein the plurality of beads is immobilized to the top surface.

53. The system of claim 52, wherein a bead of the plurality of beads comprises a plurality of amplified nucleic acid molecules comprising a substantially identical sequence.

54. The system of claim 49, wherein the substrate is substantially planar.

55. The system of claim 49, wherein the substrate is unpatterned.

56. The system of claim 49, wherein the substrate is patterned.

57. The system of claim 38, further comprising one or more processors operably connected to the fluid dispenser, the detector, and the chuck, and individually or collectively programmed to rotate the substrate via the chuck, dispense fluid via the fluid dispenser, or collect signals from the substrate via the detector.
